(19) United States
(12) Patent Application Publication
(10) Pub. No.: US 2022/0378072 A1
(43) Pub. Date: Dec. 1, 2022

(54) BIOSYNTHESIS OF MOGROSIDES

(71) Applicant: Ginkgo Bioworks, Inc., Boston, MA (US)

(72) Inventors: Jeffrey Ian Boucher, (US); Matthew McMahon, (US); Scott Marr, (US); Jie Zhu, (US)

(73) Assignee: Ginkgo Bioworks, Inc., Boston, MA (US)

(21) Appl. No.: 17/771,146

(22) PCT Filed: Oct. 23, 2020
(86) PCT No.: PCT/US2020/057067

§ 371 Date: Apr. 22, 2022

Related U.S. Application Data
(60) Provisional application No. 62/926,170, filed on Oct. 25, 2019.

(51) Int. Cl. A23L 27/30; C07K 14/415; C12N 15/62; C12N 9/02; C12N 9/10; C12N 9/24; C12N 9/90

(52) U.S. Cl. CPC A23L 27/36; C07K 14/415; C12N 15/62; C12N 9/0042; C12N 9/0071; C12N 9/1051; C12N 9/24; C12N 9/90; C12Y 106/02004; C12Y 114/14; C12Y 303/02009; C12Y 504/99033; C07K 2319/02; C07K 2319/03

(57) ABSTRACT

The disclosure relates to enzymes, such as cucurbitadienol synthase (CDS), UDP-glycosyltransferase (UGT), C11 hydroxylase, epoxide hydrolase (EPH), squalene epoxidase (SQE), and/or cytochrome P450 reductase enzymes, recombinant host cells expressing the enzymes, and methods of producing mogrol precursors, mogrol, and/or mogrosides using such recombinant cells.

### CROSS-REFERENCE TO RELATED APPLICATIONS

This application claims the benefit under 35 U.S.C. § 119(e) of U.S. Provisional Application Ser. No. 62/926,170, filed Oct. 25, 2019, entitled “BIOSYNTHESIS OF MOGROSIDES,” the disclosure of which is incorporated by reference here in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Oct. 23, 2020, is named G091970038WO00-SEQ-FL and is 831 KB in size.

### FIELD OF THE INVENTION

The present disclosure relates to the production of mogrol precursors, mogrol and mogrosides in recombinant cells.

### BACKGROUND

Mogrosides are glycosides of cucurbitane derivatives. Highly sought after as sweeteners and sugar alternatives, mogrosides are naturally synthesized in the fruits of plants, including Siraitia grosvenorii (S. grosvenorii). Although anti-cancer, anti-oxidative, and anti-inflammatory properties have been ascribed to mogrosides, characterization of the exact enzymes involved in mogroside biosynthesis is limited. Furthermore, mogroside extraction from fruit is labor-intensive and the structural complexity of mogrosides often hinders de novo chemical synthesis.

### SUMMARY

Aspects of the present disclosure provide host cells that express a C11 hydroxylase fusion protein. In some embodiments, the C11 hydroxylase fusion protein comprises: (a) a signal sequence that comprises a sequence selected from SEQ ID NO: 226, SEQ ID NO: 220 or SEQ ID NOs: 209-219, 221-225, or 227-232 or a sequence that has no more than two amino acid substitutions, deletions, or insertions relative to a sequence selected from SEQ ID NO: 226, SEQ ID NO: 220 or SEQ ID NOs: 209-219, 221-225, or 227-232; and (b) a sequence comprising a transmembrane domain and a catalytic domain of a C11 hydroxylase enzyme.

In some embodiments, the signal sequence comprises a sequence selected from SEQ ID NO: 226, SEQ ID NO: 220 or SEQ ID NOs: 209-219, 221-225, or 227-232.

In some embodiments, the sequence in (b) comprises a sequence that is at least 90% identical to wild-type CYP5491 (SEQ ID NO: 208).

In some embodiments, the transmembrane domain of the C11 hydroxylase enzyme comprises residues that correspond to residues 2-28 of wild-type CYP5491 (SEQ ID NO: 208) and/or the catalytic domain of the C11 hydroxylase enzyme comprises residues that correspond to residues 29-473 of wild-type CYP5491 (SEQ ID NO: 208).

In some embodiments, the sequence comprising the catalytic domain of the C11 hydroxylase enzyme comprises an amino acid substitution, deletion, or insertion in the catalytic domain relative to the catalytic domain of wild-type CYP5491 (residues 29-473 of SEQ ID NO: 208).

In some embodiments, the amino acid substitution, deletion, or insertion is located in the substrate binding domain of the C11 hydroxylase enzyme.

In some embodiments, the amino acid substitution, deletion, or insertion is located in a loop that binds a heme group.

In some embodiments, relative to the sequence of wild-type CYP5491 (SEQ ID NO: 208), the C11 hydroxylase enzyme comprises an amino acid substitution at a residue corresponding to residues: S49; V57; L76; A85; D107; L109; F112; T117; W119; L120; A140; F147; S155; H160; K185; L210; S211; L212; A282; D299; V350; T351; A353; L354; M376; I458; and/or T470 in CYP5491.

In some embodiments, the C11 hydroxylase enzyme comprises: A, F, H, I, M, or L at the residue corresponding to S49 of wild-type CYP5491 (SEQ ID NO: 208); A at the residue corresponding to V57 of wild-type CYP5491 (SEQ ID NO: 208); I or V at the residue corresponding to L76 of wild-type CYP5491 (SEQ ID NO: 208); S at the residue corresponding to A85 of wild-type CYP5491 (SEQ ID NO: 208); P or R at the residue corresponding to D107 of wild-type CYP5491 (SEQ ID NO: 208); A, C, F, W, or Y at the residue corresponding to L109 of wild-type CYP5491 (SEQ ID NO: 208); T or W at the residue corresponding to F112 of wild-type CYP5491 (SEQ ID NO: 208); G at the residue corresponding to T117 of wild-type CYP5491 (SEQ ID NO: 208); R at the residue corresponding to W119 of wild-type CYP5491 (SEQ ID NO: 208); H or N at the residue corresponding to L120 of wild-type CYP5491 (SEQ ID NO: 208); P at the residue corresponding to A140 of wild-type CYP5491 (SEQ ID NO: 208); L at the residue corresponding to F147 of wild-type CYP5491 (SEQ ID NO: 208); A at the residue corresponding to S155 of wild-type CYP5491 (SEQ ID NO: 208); E at the residue corresponding to H160 of wild-type CYP5491 (SEQ ID NO: 208); H at the residue corresponding to K185 of wild-type CYP5491 (SEQ ID NO: 208); S at the residue corresponding to L210 of wild-type CYP5491 (SEQ ID NO: 208); N at the residue corresponding to S211 of wild-type CYP5491 (SEQ ID NO: 208); F at the residue corresponding to L212 of wild-type CYP5491 (SEQ ID NO: 208); V at the residue corresponding to A282 of wild-type CYP5491 (SEQ ID NO: 208); A at the residue corresponding to D299 of wild-type CYP5491 (SEQ ID NO: 208); F, I, L, or M at the residue corresponding to V350 of wild-type CYP5491 (SEQ ID NO: 208); L or M at the residue corresponding to T351 of wild-type CYP5491 (SEQ ID NO: 208); G at the residue corresponding to A353 of wild-type CYP5491 (SEQ ID NO: 208); V or I at the residue corresponding to L354 of wild-type CYP5491 (SEQ ID NO: 208); A or C at the residue corresponding to M376 of wild-type CYP5491 (SEQ ID NO: 208); P at the residue corresponding to I458 of wild-type CYP5491 (SEQ ID NO: 208); and/or E at the residue corresponding to T470 of wild-type CYP5491 (SEQ ID NO: 208).

In some embodiments, the host cell further comprises an upregulated squalene epoxidase (SQE), at least one cytochrome P450 reductase, at least one cucurbitadienol synthase (CDS), and/or at least one epoxide hydrolase (EPH).

In some embodiments, the host cell comprises an upregulated squalene synthase, a downregulated lanosterol synthase, at least one other C11 hydroxylase, and/or at least two cytochrome P450 reductases.

In some embodiments, a nucleotide sequence encoding the C11 hydroxylase fusion protein is integrated into the genome of the host cell. In some embodiments, a nucleotide sequence encoding the C11 hydroxylase fusion protein is expressed on a plasmid.

In some embodiments, multiple copies of a nucleotide sequence encoding the C11 hydroxylase fusion protein are integrated into the genome of the host cell.

In some embodiments, at least one nucleotide sequence encoding the squalene synthase, the squalene epoxidase, the at least one other C11 hydroxylase, the at least one cytochrome P450 reductase, the at least one CDS, and/or the at least one EPH is integrated into the genome of the host cell.

In some embodiments, the host cell produces mogrol.

In some embodiments, the host cell produces at least 1.1 fold more mogrol compared to a control host cell, wherein the control host cell comprises wild-type CYP5491.

In some embodiments, the host cell is capable of being cultured in cell culture media that is substantially free of one or more mogrol precursors that are not produced by the host cell.

In some embodiments, the host cell is capable of producing a ratio of mogrol/11-oxomogrol that is greater than 2.

In some embodiments, the C11 hydroxylase fusion protein comprises a sequence that is at least 90% identical to a sequence selected from SEQ ID NO: 305 or 308, SEQ ID NOs: 257-280, or SEQ ID NOs: 306-307, or SEQ ID NOs: 309-310.

Further aspects of the present disclosure provide host cells that comprise a C11 hydroxylase enzyme, wherein the C11 hydroxylase enzyme is at least 90% identical to wild-type CYP5491 (SEQ ID NO: 208) and comprises an amino acid substitution at one or more residues corresponding to residues in CYP5491. In some embodiments, the one or more residues correspond to residues: S49; V57; L76; A85; D107; L109; F112; T117; W119; L120; A140; F147; S155; H160; K185; L210; S211; L212; A282; D299; V350; T351; A353; L354; M376; I458; and/or T470 in CYP5491.

In some embodiments, the C11 hydroxylase enzyme comprises: A, F, H, I, M, or L at the residue corresponding to S49 of wild-type CYP5491 (SEQ ID NO: 208); A at the residue corresponding to V57 of wild-type CYP5491 (SEQ ID NO: 208); I or V at the residue corresponding to L76 of wild-type CYP5491 (SEQ ID NO: 208); S at the residue corresponding to A85 of wild-type CYP5491 (SEQ ID NO: 208); P or R at the residue corresponding to D107 of wild-type CYP5491 (SEQ ID NO: 208); A, C, F, W, or Y at the residue corresponding to L109 of wild-type CYP5491 (SEQ ID NO: 208); T or W at the residue corresponding to F112 of wild-type CYP5491 (SEQ ID NO: 208); G at the residue corresponding to T117 of wild-type CYP5491 (SEQ ID NO: 208); R at the residue corresponding to W119 of wild-type CYP5491 (SEQ ID NO: 208); H or N at the residue corresponding to L120 of wild-type CYP5491 (SEQ ID NO: 208); P at the residue corresponding to A140 of wild-type CYP5491 (SEQ ID NO: 208); L at the residue corresponding to F147 of wild-type CYP5491 (SEQ ID NO: 208); A at the residue corresponding to S155 of wild-type CYP5491 (SEQ ID NO: 208); E at the residue corresponding to H160 of wild-type CYP5491 (SEQ ID NO: 208); H at the residue corresponding to K185 of wild-type CYP5491 (SEQ ID NO: 208); S at the residue corresponding to L210 of wild-type CYP5491 (SEQ ID NO: 208); N at the residue corresponding to S211 of wild-type CYP5491 (SEQ ID NO: 208); F at the residue corresponding to L212 of wild-type CYP5491 (SEQ ID NO: 208); V at the residue corresponding to A282 of wild-type CYP5491 (SEQ ID NO: 208); A at the residue corresponding to D299 of wild-type CYP5491 (SEQ ID NO: 208); F, I, L, or M at the residue corresponding to V350 of wild-type CYP5491 (SEQ ID NO: 208); L or M at the residue corresponding to T351 of wild-type CYP5491 (SEQ ID NO: 208); G at the residue corresponding to A353 of wild-type CYP5491 (SEQ ID NO: 208); V or I at the residue corresponding to L354 of wild-type CYP5491 (SEQ ID NO: 208); A or C at the residue corresponding to M376 of wild-type CYP5491 (SEQ ID NO: 208); P at the residue corresponding to I458 of wild-type CYP5491 (SEQ ID NO: 208); and/or E at the residue corresponding to T470 of wild-type CYP5491 (SEQ ID NO: 208).

In some embodiments, the C11 hydroxylase enzyme comprises: (a) a phenylalanine (F) or leucine (L) at a residue corresponding to S49 in wild-type CYP5491 (SEQ ID NO: 208); and/or (b) a methionine (M) at a residue corresponding to T351 in wild-type CYP5491 (SEQ ID NO: 208).

In some embodiments, the C11 hydroxylase enzyme is expressed as a C11 hydroxylase fusion protein that comprises a signal sequence that is at least 90% identical to a sequence selected from SEQ ID NO: 226, SEQ ID NO: 220 or SEQ ID NOs: 209-219, 221-225, or 227-232.

In some embodiments, the signal sequence comprises a sequence selected from SEQ ID NO: 226, SEQ ID NO: 220 or SEQ ID NOs: 209-219, 221-225, or 227-232.

In some embodiments, the host cell further comprises an upregulated squalene epoxidase, at least one cytochrome P450 reductase, at least one cucurbitadienol synthase (CDS), and/or at least one epoxide hydrolase (EPH).

In some embodiments, the host cell comprises an upregulated squalene synthase, a downregulated lanosterol synthase, at least one other C11 hydroxylase, and/or at least two cytochrome P450 reductases.

In some embodiments, a nucleotide sequence encoding the C11 hydroxylase enzyme is integrated into the genome of the host cell or a nucleotide sequence encoding the C11 hydroxylase enzyme is expressed on a plasmid.

In some embodiments, multiple copies of a nucleotide sequence encoding the C11 hydroxylase enzyme are integrated into the genome of the host cell.

In some embodiments, at least one nucleotide sequence encoding the squalene synthase, the squalene epoxidase, the at least one other C11 hydroxylase, the at least one cytochrome P450 reductase, the at least one CDS, and/or the at least one EPH is integrated into the genome of the host cell.

In some embodiments, the host cell produces mogrol.

In some embodiments, the host cell produces 1.1 fold more mogrol compared to a control host cell, wherein the control host cell comprises wild-type CYP5491.

In some embodiments, the host cell is capable of being cultured in cell culture media that is substantially free of one or more mogrol precursors that are not produced by the host cell.

In some embodiments, the host cell is capable of producing a ratio of mogrol/11-oxomogrol that is greater than 2.

Further aspects of the present disclosure provide methods of producing mogrol. In some embodiments, the methods comprise culturing any of the host cells disclosed.

In some embodiments, the host cell is cultured in the presence of a mogrol precursor selected from squalene, 2-3-oxidosqualene, cucurbitadienol, 2-3, 22,23-diepoxysqualene, 24, 25 epoxy-cucurbitadienol, and 24, 25 dihydroxy cucurbitadienol.

In some embodiments, the host cell is cultured in media that is substantially free of one or more mogrol precursors that are not produced by the host cell.

Further aspects of the present disclosure provide host cells that comprise a C11 hydroxylase fusion protein, wherein the C11 hydroxylase fusion protein comprises the signal sequence of ERG11 and a sequence encoding a transmembrane domain and a catalytic domain of a C11 hydroxylase enzyme.

In some embodiments, the C11 hydroxylase fusion protein comprises residues 3-504 of wild-type CYP5491 (SEQ ID NO: 208).

In some embodiments, the C11 hydroxylase fusion protein comprises a sequence that is at least 90% identical to SEQ ID NO: 280.

Further aspects of the present disclosure provide C11 hydroxylase fusion proteins, wherein the fusion protein comprises: (a) a signal sequence that is at least 90% identical to a sequence selected from SEQ ID NO: 226, SEQ ID NO: 220 or SEQ ID NOs: 209-219, 221-225, or 227-232, and a sequence encoding a transmembrane domain and a catalytic domain of a C11 hydroxylase enzyme; or (b) the first 25 amino acids of ERG11, and a sequence encoding a transmembrane domain and a catalytic domain of a C11 hydroxylase enzyme.

Further aspects of the present disclosure provide methods of producing mogrol comprising contacting a C11 hydroxylase enzyme with (a) 24, 25 dihydroxy cucurbitadienol, thereby producing mogrol; (b) cucurbitadienol, thereby producing 11-hydroxycucurbitadienol; and/or (c) 24,25-epoxy cucurbitadienol, thereby producing, 11-hydroxy-24,25-epoxycucurbitadienol, wherein the C11 hydroxylase enzyme is at least 90% identical to SEQ ID NO: 208 and comprises at least one amino acid substitution relative to SEQ ID NO: 208.

In some embodiments, relative to the sequence of wild-type CYP5491 (SEQ ID NO: 208), the C11 hydroxylase enzyme comprises an amino acid substitution at a residue corresponding to residues: S49; V57; L76; A85; D107; L109; F112; T117; W119; L120; A140; F147; S155; H160; K185; L210; S211; L212; A282; D299; V350; T351; A353; L354; M376; I458; and/or T470 in wild-type CYP5491 (SEQ ID NO: 208).

In some embodiments, the C11 hydroxylase enzyme comprises: A, F, H, I, M, or L at the residue corresponding to S49 in wild-type CYP5491 (SEQ ID NO: 208); A at the residue corresponding to V57 in wild-type CYP5491 (SEQ ID NO: 208); I or V at the residue corresponding to L76 in wild-type CYP5491 (SEQ ID NO: 208); S at the residue corresponding to A85 in wild-type CYP5491 (SEQ ID NO: 208); P or R at the residue corresponding to D107 in wild-type CYP5491 (SEQ ID NO: 208); A, C, F, W, or Y at the residue corresponding to L109 in wild-type CYP5491 (SEQ ID NO: 208); T or W at the residue corresponding to F112 in wild-type CYP5491 (SEQ ID NO: 208); G at the residue corresponding to T117 in wild-type CYP5491 (SEQ ID NO: 208); Rat the residue corresponding to W119 in wild-type CYP5491 (SEQ ID NO: 208); H or N at the residue corresponding to L120 in wild-type CYP5491 (SEQ ID NO: 208); P at the residue corresponding to A140 in wild-type CYP5491 (SEQ ID NO: 208); L at the residue corresponding to F147 in wild-type CYP5491 (SEQ ID NO: 208); A at the residue corresponding to S155 in wild-type CYP5491 (SEQ ID NO: 208); E at the residue corresponding to H160 in wild-type CYP5491 (SEQ ID NO: 208); H at the residue corresponding to K185 in wild-type CYP5491 (SEQ ID NO: 208); S at the residue corresponding to L210 in wild-type CYP5491 (SEQ ID NO: 208); N at the residue corresponding to S211 in wild-type CYP5491 (SEQ ID NO: 208); F at the residue corresponding to L212 in wild-type CYP5491 (SEQ ID NO: 208); V at the residue corresponding to A282 in wild-type CYP5491 (SEQ ID NO: 208); A at the residue corresponding to D299 in wild-type CYP5491 (SEQ ID NO: 208); F, I, L, or M at the residue corresponding to V350 in wild-type CYP5491 (SEQ ID NO: 208); L or M at the residue corresponding to T351 in wild-type CYP5491 (SEQ ID NO: 208); G at the residue corresponding to A353 in wild-type CYP5491 (SEQ ID NO: 208); V or I at the residue corresponding to L354 in wild-type CYP5491 (SEQ ID NO: 208); A or C at the residue corresponding to M376 in wild-type CYP5491 (SEQ ID NO: 208); P at the residue corresponding to I458 in wild-type CYP5491 (SEQ ID NO: 208); and/or E at the residue corresponding to T470 in wild-type CYP5491 (SEQ ID NO: 208).

In some embodiments, the C11 hydroxylase enzyme is a purified protein.

Further aspects of the disclosure provide host cells that comprise a C11 hydroxylase fusion protein, wherein the C11 hydroxylase fusion protein comprises: the signal sequence of KAR2, NCP1, ERP2, RBD2, SNA3, SPC2, NHX1, PGA2, GRX6, YLR413W, YJL062W, MSC2, EMCS, CHO2, IFA38, SUR2, IPT1, YET3, YPL162C, ERG11, SRP102, GUP1, CBR1, or YHR138C; and a sequence encoding a transmembrane domain and a catalytic domain of a C11 hydroxylase enzyme.

Further aspects of the disclosure provide host cells comprising a C11 hydroxylase fusion protein, wherein the C11 hydroxylase fusion protein is at least 90% identical to any one of SEQ ID NO: 305 or 308, SEQ ID NOs: 257-280, or SEQ ID NOs: 306-307, or SEQ ID NOs: 309-310. In some embodiments, the C11 hydroxylase fusion protein is at least 98% identical to any one of SEQ ID NO: 305 or 308, SEQ ID NOs: 257-280, or SEQ ID NOs: 306-307, or SEQ ID NOs: 309-310. In some embodiments, the C11 hydroxylase fusion protein is at least 99% identical to any one of SEQ ID NO: 305 or 308, SEQ ID NOs: 257-280, or SEQ ID NOs: 306-307, or SEQ ID NOs: 309-310.

In some embodiments, the C11 hydroxylase comprises any one of SEQ ID NO: 305 or 308, SEQ ID NOs: 257-280, or SEQ ID NOs: 306-307, or SEQ ID NOs: 309-310. In some embodiments, the C11 hydroxylase is PGA2Id23-129_CYP5491-T351M (SEQ ID NO: 305).

Further aspects of the disclosure provide methods comprising culturing any of the host cells disclosed.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used in this disclosure is for the purpose of description and should not be regarded as limiting. The use of “including,” “comprising,” or “having,” “containing,” “involving,” and variations thereof, is meant to encompass the items listed thereafter and equivalents thereof as well as additional items.

### BRIEF DESCRIPTION OF DRAWINGS

The accompanying drawings are not intended to be drawn to scale. The drawings are illustrative only and are not required for enablement of the disclosure. For purposes of clarity, not every component may be labeled in every drawing. In the drawings:

FIGS. 1A-1B are schematic overviews of putative mogrol biosynthesis pathways. SQS indicates squalene synthase, EPD indicates Epoxidase, P450 indicates C11 hydroxylase, EPH indicates epoxide hydrolase, and CDS indicates cucurbitadienol synthase.

FIGS. 2A-2C depict schematics showing: the generalized structure of a C11 hydroxylase; development of a C11 hydroxylase fusion protein library based on the C11 hydroxylase CYP5491; and design of the C11 hydroxylase library plasmid. FIG. 2A depicts the structure of a C11 hydroxylase. FIG. 2B is a schematic showing development of a C11 hydroxylase fusion protein library. FIG. 2C is a schematic showing the P450 library plasmid structure.

FIG. 3 depicts the development of a host cell for screening CYP5491 fusion proteins and CYP5491 mutants. FIG. 3 shows that integration of multiple copies of wild-type CYP5491 into the genome of the screening strain promotes mogrol production, with one transformant, P450 Base strain-3, of the strain showing a higher titer than the rest.

FIGS. 4A-4B depict diagrams showing residues in the active site of CYP5491 that are in close proximity to a modeled lanosterol and that were subjected to saturation mutagenesis and screening.

FIGS. 5A-5B depict graphs showing the production of mogrol by host cells encoding a member of the C11 hydroxylase screening library relative to mogrol production of control host cells encoding wild-type CYP5491. FIG. 5A shows the results for all members of the screening library as compared to the control strain (t401172) that encodes multiple copies of wild-type CYP5491. FIG. 5B is a magnification of a portion of the graph depicted in FIG. 5A.

FIG. 6 is a graph showing results of mogrol production from a secondary validation screen of 71 hits from the C11 hydroxylase screening library. The results from the primary screen are also shown for comparison. The y-axis shows fold improvement in mogrol production relative to the control host cells encoding multiple copies of wild-type CYP5491.

FIG. 7 depicts a graph showing fold improvement of mogrol production with host cells comprising the indicated CYP5491 mutants, signal peptide-wild-type CYP5491 fusion proteins, and signal peptide-CYP5491 mutant fusion proteins compared to wild-type CYP5491 (SEQ ID NO: 208) (shown left-most).

FIG. 8 depicts a graph showing the mean mogrol production (mg/L), mean oxomogrol production (mg/L), and mean mogrol/oxo mogrol ratios of host cells comprising the indicated wild-type CYP5491, CYP5491 mutants, signal peptide-wild-type CYP5491 fusion proteins, and signal peptide-CYP5491 mutant fusion proteins. Results for a control strain are also shown.

### DETAILED DESCRIPTION

Mogrosides are widely used as natural sweeteners, for example in beverages. However, de novo synthesis and mogroside extraction from natural sources often involves high production costs and low yield. This application describes recombinant host cells that are engineered to efficiently produce mogrol (or 11, 24, 25-trihydroxy cucurbitadienol), mogrosides, and precursors thereof. Methods include heterologous expression of cucurbitadienol synthase (CDS) enzymes, UDP-glycosyltransferase (UGT) enzymes, C11 hydroxylase enzymes, cytochrome P450 reductase enzymes, epoxide hydrolase (EPH) enzymes, squalene epoxidase (SQE) enzymes, or combinations thereof. This application describes the identification of improved C11 hydroxylases for mogrol production. Examples 1-2 describe screening of C11 hydroxylases, including variants and fusion proteins, resulting in identification of C11 hydroxylases with improved mogrol production relative to wild-type C11 hydroxylase CYP5491. Enzymes and recombinant host cells described in this application can be used for making mogrol, mogrosides, and precursors thereof.

### Synthesis of Mogrol and Mogrosides

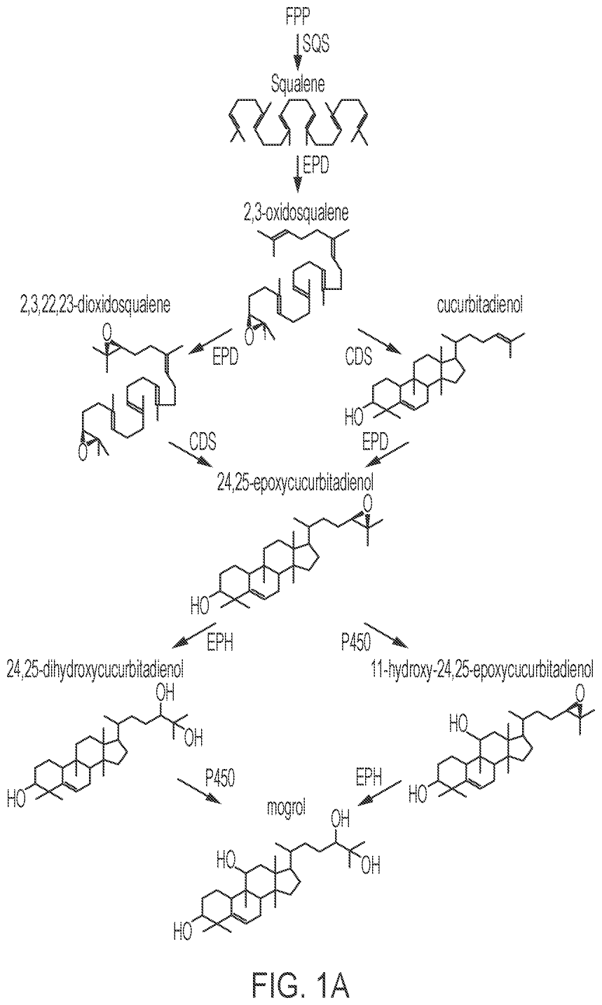

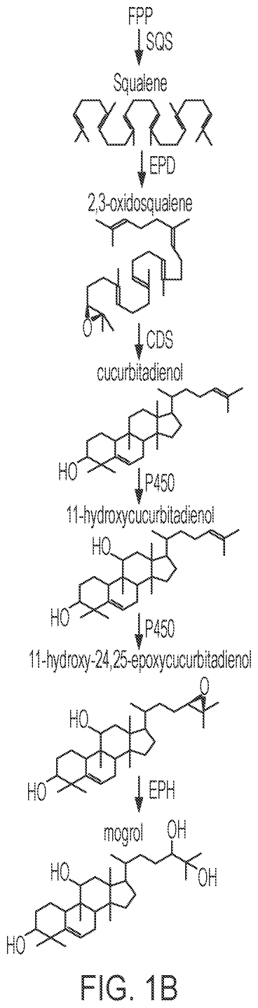

FIGS. 1A-1B show a putative mogrol synthesis pathway. An early step in the pathway involves conversion of squalene to 2,3-oxidoqualene. As shown in FIG. 1A, 2,3-oxidosqualene is first cyclized to cucurbitadienol followed by epoxidation to form 24,25-epoxycucurbitadienol, or 2,3-oxidosqualene is epoxidized to 2,3,22,23-dioxidosqualene and then cyclized to 24,25-epoxycucurbitadienol. Next, the 24,25-epoxycucurbitadienol can be converted to mogrol (aglycone of mogrosides) following epoxide hydrolysis and then oxidation, or oxidation and then epoxide hydrolysis. As shown in FIG. 1B, 2,3-oxidosqualene is first cyclized to cucurbitadienol, which is then converted to 11-hydroxycucurbitadienol by a cytochrome P450 C11 hydroxylase. Then, a cytochrome P450 C11 hydroxylase may convert 11-hydroxycucurbitadienol to 11-hydroxy-24,25-epoxycucurbitadienol. 11-hydroxy-24,25-epoxycucurbitadienol may be converted to mogrol by epoxide hydrolase. C11 hydroxylases act in conjunction with cytochrome P450 reductases (not shown in FIGS. 1A-1B).

Mogrol can be distinguished from other cucurbitane triterpenoids by oxygenations at C3, C11, C24, and C25. Glycosylation of mogrol, for example at C3 and/or C24 leads to the formation of mogrosides.

Mogrol precursors include but are not limited to squalene, 2-3-oxidosqualene, 2,3,22,23-dioxidosqualene, cucurbitadienol, 24, 25-expoxycucurbitadienol, 11-hydroxycucurbitadienol, 11-hydroxy-24,25-epoxycucurbitadienol, 11-hydroxy-cucurbitadienol, 11-oxo-cucurbitadienol, and 24,25-dihydroxycucurbitadienol. The term “dioxidosqualene” may be used to refer to 2,3,22,23-diepoxy squalene or 2,3,22,23-dioxido squalene. The term “2,3-epoxysqualene” may be used interchangeably with the term “2-3-oxidosqualene.” As used in this application, mogroside precursors include mogrol precursors, mogrol and mogrosides.

Examples of mogrosides include, but are not limited to, mogroside I-A1 (MIA1), mogroside IE (MIE), mogroside II-A1 (MIIA1), mogroside II-A2 (MIIA2), mogroside III-A1 (MIIIA1), mogroside II-E (MIIE), mogroside III (MIIIE), siamenoside I, mogroside IV, mogroside IVa, isomogroside IV, mogroside III-E (MIIIE), mogroside V, and mogroside VI.

In some embodiments, the mogroside produced is siamenoside I, which may be referred to as Siam. In some embodiments, the mogroside produced is MIIIE.

In other embodiments, a mogroside is a compound of Formula 1:

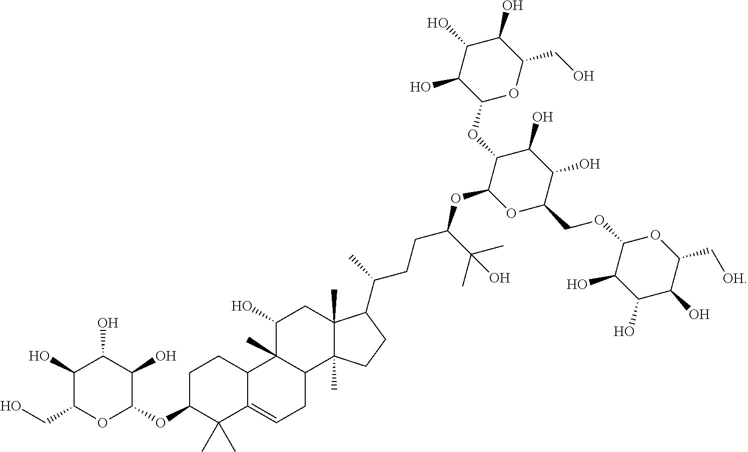 [SMILES: C[C@H](CC[C@@H](O[C@@H]1O[C@H](CO[C@@H]2O[C@H](CO)[C@@H](O)[C@H](O)[C@H]2O)[C@@H](O)[C@H](O)[C@H]1O[C@H]1O[C@@H](CO)[C@H](O)[C@@H](O)[C@@H]1O)C(C)(C)O)C1CC[C@@]2(C)C3CC=C4C(CC[C@H](O[C@@H]5O[C@H](CO)[C@@H](O)[C@H](O)[C@H]5O)C4(C)C)[C@]3(C)[C@H](O)C[C@]12C]

In some embodiments, the methods described in this application may be used to produce any of the compounds described in and incorporated by reference from US 2019/0071705, including compounds 1-20 as disclosed in US 2019/0071705. In some embodiments, the methods described in this application may be used to produce variants of any of the compounds described in and incorporated by reference from US 2019/0071705, including variants of compounds 1-20 as disclosed in US 2019/0071705. For example, a variant of a compound described in US 2019/0071705 can comprise a substitution of one or more alpha-glucosyl linkages in a compound described in US 2019/0071705 with one or more beta-glucosyl linkages. In some embodiments, a variant of a compound described in US 2019/0071705 comprises a substitution of one or more beta-glucosyl linkages in a compound described in US 2019/0071705 with one or more alpha-glucosyl linkages. In some embodiments, a variant of a compound described in US 2019/0071705 is a compound of Formula 1 show above.

C11 hydroxylase Enzymes

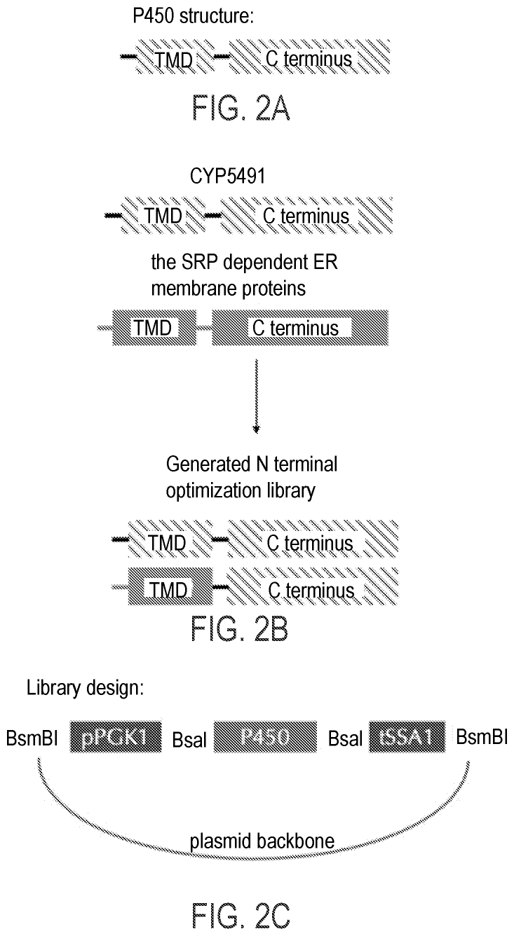

Aspects of the disclosure provide C11 hydroxylases, which may be useful, for example, in the production of mogrol. C11 hydroxylases are a type of P450 enzyme. As used in this disclosure, a C11 hydroxylase refers to an enzyme that is capable of introducing a hydroxyl group into the C11 position of a compound. In some embodiments, the compound is a mogrol precursor. In some embodiments, the compound is a mogroside. In some embodiments, a C11 hydroxylase is also capable of introducing a hydroxyl group into a position that is not C11 on a compound. In some embodiments, a C11 hydroxylase is a C11 hydroxylase that is capable of catalyzing C11 hydroxylation of a mogrol precursor. In some embodiments, the mogrol precursor is 24,25-epoxycucurbitadienol or 24,25-dihydroxycucurbitadienol. In some embodiments, a C11 hydroxylase is capable of producing 11-hydroxy-24,25-epoxycucurbitadienol. In some embodiments, a C11 hydroxylase is capable of producing mogrol. In some embodiments, a C11 hydroxylase is capable of producing 11-oxo mogrol. In some embodiments, a mogrol precursor is 11-hydroxy-cucurbitadienol. In some embodiments, a mogrol precursor is 11-oxo-cucurbitadienol. In some embodiments, a C11 hydroxylase uses cucurbitadienol as a substrate to produce 11-hydroxy-cucurbitadienol. In some embodiments, a C11 hydroxylase uses 11-hydroxy-cucurbitadienol as a substrate to produce 11-oxo cucurbitadienol. In some embodiments, a C11 hydroxylase uses cucurbitadienol as a substrate to produce 11-hydroxy-cucurbitadienol and then converts 11-hydroxy-cucurbitadienol to 11-oxo cucurbitadienol. Structurally, C11 hydroxylases generally comprise a transmembrane domain and a catalytic domain, as shown in FIG. 2A. As used in this application, a “transmembrane domain” refers to a domain encompassing the membrane-spanning region of a protein. As used in this application, a “catalytic domain” refers to a region of an enzyme comprising the active site. In some embodiments, the catalytic domain of a C11 hydroxylase can bind heme. In some embodiments, a C11 hydroxylase is localized to the endoplasmic reticulum (ER).

An example of a C11 hydroxylase is CYP5491. A non-limiting example of a nucleotide sequence encoding wild-type CYP5491 is:

(SEQ ID NO: 207)
ATGTGGACTGTCGTGCTCGGTTTGGCGACGCTGTTTGTCGCCTACTACAT
CCATTGGATTAACAAATGGAGAGATTCCAAGTTCAACGGAGTTCTGCCGC
CGGGCACCATGGGTTTGCCGCTCATCGGAGAAACGATTCAACTGAGTCGA
CCCAGTGACTCCCTCGACGTTCACCCTTTCATCCAGAAAAAAGTTGAAAG
ATACGGGCCGATCTTCAAAACATGTCTGGCCGGAAGGCCGGTGGTGGTGT
CGGCGGACGCAGAGTTCAACAACTACATAATGCTGCAGGAAGGAAGAGCA
GTGGAAATGTGGTATTTGGATACGCTCTCCAAATTTTTCGGCCTCGACAC
CGAGTGGCTCAAAGCTCTGGGCCTCATCCACAAGTACATCAGAAGCATTA
CTCTCAATCACTTCGGCGCCGAGGCCCTGCGGGAGAGATTTCTTCCTTTT
ATTGAAGCATCCTCCATGGAAGCCCTTCACTCCTGGTCTACTCAACCTAG
CGTCGAAGTCAAAAATGCCTCCGCTCTCATGGTTTTTAGGACCTCGGTGA
ATAAGATGTTCGGTGAGGATGCGAAGAAGCTATCGGGAAATATCCCTGGG
AAGTTCACGAAGCTTCTAGGAGGATTTCTCAGTTTACCACTGAATTTTCC
CGGCACCACCTACCACAAATGCTTGAAGGATATGAAGGAAATCCAGAAGA
AGCTAAGAGAGGTTGTAGACGATAGATTGGCTAATGTGGGCCCTGATGTG
GAAGATTTCTTGGGGCAAGCCCTTAAAGATAAGGAATCAGAGAAGTTCAT
TTCAGAGGAGTTCATCATCCAACTGTTGTTTTCTATCAGTTTTGCTAGCT
TTGAGTCCATCTCCACCACTCTTACTTTGATTCTCAAGCTCCTTGATGAA
CACCCAGAAGTAGTGAAAGAGTTGGAAGCTGAACACGAGGCGATTCGAAA
AGCTAGAGCAGATCCAGATGGACCAATTACTTGGGAAGAATACAAATCCA
TGACTTTTACATTACAAGTCATCAATGAAACCCTAAGGTTGGGGAGTGTC
ACACCTGCCTTGTTGAGGAAAACAGTTAAAGATCTTCAAGTAAAAGGATA
CATAATCCCGGAAGGATGGACAATAATGCTTGTCACCGCTTCACGTCACA
GAGATCCAAAAGTCTATAAGGACCCTCATATCTTCAATCCATGGCGTTGG
AAGGACTTGGACTCAATTACCATCCAAAAGAACTTCATGCCTTTTGGGGG
AGGCTTAAGGCATTGTGCTGGTGCTGAGTACTCTAAAGTCTACTTGTGCA
CCTTCTTGCACATCCTCTGTACCAAATACCGATGGACCAAACTTGGGGGA
GGAAGGATTGCAAGAGCTCATATATTGAGTTTTGAAGATGGGTTACATGT
GAAGTTCACGCCAAAAGAATAA

The amino acid sequence of wild-type CYP5491 is:

(SEQ ID NO: 208)
MWTVVLGLATLFVAYYIHWINKWRDSKFNGVLPPGTMGLPLIGETIQLSR
PSDSLDVHPFIQKKVERYGPIFKTCLAGRPVVVSADAEFNNYIMLQEGRA
VEMWYLDTLSKFFGLDTEWLKALGLIHKYIRSITLNHFGAEALRERFLPF
IEASSMEALHSWSTQPSVEVKNASALMVFRTSVNKMFGEDAKKLSGNIPG
KFTKLLGGFLSLPLNFPGTTYHKCLKDMKEIQKKLREVVDDRLANVGPDV
EDFLGQALKDKESEKFISEEFIIQLLFSISFASFESISTTLTLILKLLDE
HPEVVKELEAEHEAIRKARADPDGPITWEEYKSMTFTLQVINETLRLGSV
TPALLRKTVKDLQVKGYIIPEGWTIMLVTASRHRDPKVYKDPHIFNPWRW
KDLDSITIQKNFMPFGGGLRHCAGAEYSKVYLCTFLHILCTKYRWTKLGG
GRIARAHILSFEDGLHVKFTPKE

In SEQ ID NO: 208 depicted above, underlined residues 2-28 correspond to the transmembrane domain of wild-type CYP5491. Residues 29-473, shown in bold, correspond to the catalytic domain of CYP5491.

One of ordinary skill in the art would be able to identify the transmembrane domain and/or catalytic domain of another C11 hydroxylase by using the wild-type CYP5491 amino acid sequence as a reference sequence. For example, one of ordinary skill in the art could identify the transmembrane domain and/or catalytic domain of another C11 hydroxylase by aligning the sequence of the C11 hydroxylase with the sequence of wild-type CYP5491 and identifying the residues in the C11 hydroxylase that correspond to the relevant domain in the wild-type CYP5491 sequence.

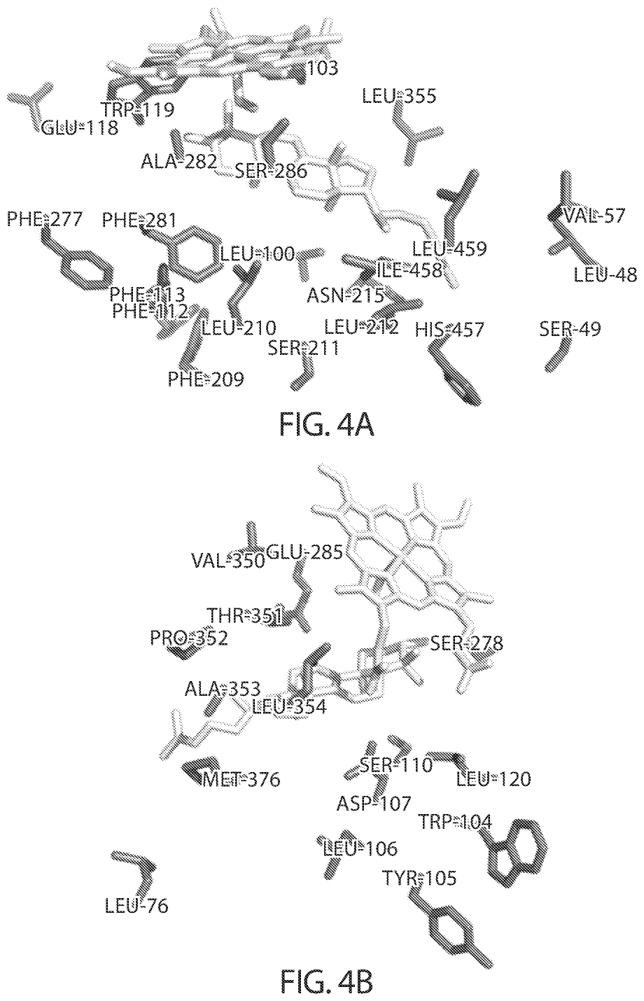

In some embodiments, a C11 hydroxylase is CYP5491. CYP5491 may have a mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 residues relative to wild-type CYP5491 (SEQ ID NO: 208). In some embodiments, the mutation is an amino acid substitution. In some embodiments, the mutation is located in one or more residues selected from residues: 48, 49, 57, 76, 103-107, 109, 110, 112, 113, 118-120, 209-212, 215, 277, 278, 281, 282, 285, 286, 350-355, 376, and/or 457-459 of CYP5491, as depicted in FIGS. 4A-4B.

In some embodiments, one or more mutations are located in a region corresponding to the substrate binding domain of a C11 hydroxylase. In some instances, one or more mutations are located in a structural loop in a C11 hydroxylase that binds a heme group. The loop that binds a heme group may comprise amino acid residues corresponding to residues 350-355 in SEQ ID NO: 208. In some instances, one or more residues corresponding to residues 281, 282, 285, 286, and 350-355 in SEQ ID NO: 208 are mutated. In some instances, the mutation is an amino acid substitution. In some instances, the mutation is a deletion. In some instances, the mutation is an insertion.

In some embodiments, a sequence encoding a C11 hydroxylase enzyme comprises an amino acid substitution at a residue corresponding to S49; V57; L76; A85; D107; L109; F112; T117; W119; L120; A140; F147; S155; H160; K185; L210; S211; L212; A282; D299; V350; T351; A353; L354; M376; I458; and/or T470 in wild-type CYP5491 (SEQ ID NO: 208). In some embodiments, a sequence encoding a C11 hydroxylase enzyme comprises: A, F, H, I, M, or L at the residue corresponding to S49 in wild-type CYP5491 (SEQ ID NO: 208); A at the residue corresponding to V57 in wild-type CYP5491 (SEQ ID NO: 208); I or V at the residue corresponding to L76 in wild-type CYP5491 (SEQ ID NO: 208); S at the residue corresponding to A85 in wild-type CYP5491 (SEQ ID NO: 208); P or R at the residue corresponding to D107 in wild-type CYP5491 (SEQ ID NO: 208); A, C, F, W, or Y at the residue corresponding to L109 in wild-type CYP5491 (SEQ ID NO: 208); T or W at the residue corresponding to F112 in wild-type CYP5491 (SEQ ID NO: 208); G at the residue corresponding to T117 in wild-type CYP5491 (SEQ ID NO: 208); R at the residue corresponding to W119 in wild-type CYP5491 (SEQ ID NO: 208); H or N at the residue corresponding to L120 in wild-type CYP5491 (SEQ ID NO: 208); P at the residue corresponding to A140 in wild-type CYP5491 (SEQ ID NO: 208); L at the residue corresponding to F147 in wild-type CYP5491 (SEQ ID NO: 208); A at the residue corresponding to S155 in wild-type CYP5491 (SEQ ID NO: 208); E at the residue corresponding to H160 in wild-type CYP5491 (SEQ ID NO: 208); H at the residue corresponding to K185 in wild-type CYP5491 (SEQ ID NO: 208); S at the residue corresponding to L210 in wild-type CYP5491 (SEQ ID NO: 208); N at the residue corresponding to S211 in wild-type CYP5491 (SEQ ID NO: 208); F at the residue corresponding to L212 in wild-type CYP5491 (SEQ ID NO: 208); V at the residue corresponding to A282 in wild-type CYP5491 (SEQ ID NO: 208); A at the residue corresponding to D299 in wild-type CYP5491 (SEQ ID NO: 208); F, I, L, or M at the residue corresponding to V350 in wild-type CYP5491 (SEQ ID NO: 208); L or M at the residue corresponding to T351 in wild-type CYP5491 (SEQ ID NO: 208); G at the residue corresponding to A353 in wild-type CYP5491 (SEQ ID NO: 208); V or I at the residue corresponding to L354 in wild-type CYP5491 (SEQ ID NO: 208); A or C at the residue corresponding to M376 in wild-type CYP5491 (SEQ ID NO: 208); P at the residue corresponding to I458 in wild-type CYP5491 (SEQ ID NO: 208); and/or E at the residue corresponding to T470 in wild-type CYP5491 (SEQ ID NO: 208).

Membrane insertion of C11 hydroxylases can be directed by an internal uncleaved signal anchor sequence located near the N terminus. Generally, C11 hydroxylases have an Nₗᵤₘᵢₙₐₗ−C_{cytosol} orientation in the ER membrane, whereby the signal-anchor sequence inserts into the ER membrane with its N-terminus facing to the lumen. The topology of C11 hydroxylases can be determined by both internal hydrophobic signal-anchor sequences and the flanking hydrophilic residues. Without being bound by any particular theory, the internal hydrophobic signal-anchor sequence (transmembrane domain) can function as an ER signal sequence, a stop-transfer sequence, and/or a membrane-anchor sequence. The membrane orientation of a C11 hydroxylase may result, at least in part, from the hydrophilic amino acids flanking the internal signal-anchor sequences. In some instances, the flanking segment that carries the greatest net positive charge remains on the cytosolic face of the membrane. The membrane orientation of proteins with internal signal-anchor sequences may also be influenced, at least in part, by the length and amino acid composition of the internal hydrophobic segment. For example, in some instances, proteins with long hydrophobic segments (>20 residues) tend to adopt a Nₗᵤₘᵢₙₐₗ−C_{cytosol} orientation, while the opposite orientation is preferred in some instances by proteins with short hydrophobic segments.

Without being bound by any particular theory, the N-terminus of plant C11 hydroxylases in some instances may be involved in directing the correct anchoring of the nascent polypeptides. In other host cells, including S. cerevisiae, however, in some instances, the N-terminus of plant C11 hydroxylases may not be sufficient to target the C11 hydroxylase to the ER.

As disclosed in this application, the activity of heterologous C11 hydroxylases in host cells, including yeast cells, may be improved by replacing the native N-terminal sequence with sequences from other C11 hydroxylases or ER-membrane bound proteins to facilitate correct folding and anchoring.

In some embodiments, a C11 hydroxylase of the present disclosure is a “C11 hydroxylase fusion.” The phrase “C11 hydroxylase fusion” is used interchangeably in this application with the phrase “C11 hydroxylase fusion protein” and refers to a C11 hydroxylase, or a portion thereof, that is fused to another sequence. Non-limiting configurations of C11 hydroxylase fusions are depicted in FIG. 2B. For example, an N-terminal region of an ER membrane protein that may or may not be endogenously expressed in a host cell may be fused to a portion of a C11 hydroxylase protein of interest. In some embodiments, a region comprising the amino acid residues that are N-terminal to the transmembrane domain of an ER membrane protein is fused to a transmembrane domain and/or a C-terminal domain of a C11 hydroxylase protein. In some embodiments, a region comprising the amino acid residues that are N-terminal to the transmembrane domain, and the transmembrane domain, of an ER membrane protein is fused to a catalytic domain of a C11 hydroxylase protein of interest. It should be understood that the terms “transmembrane domain” and “catalytic domain” in the context of a fusion protein may refer to the entire transmembrane domain or catalytic domain of a reference protein or to a portion or fragment of the transmembrane domain or catalytic domain of a reference protein.

In some embodiments, a C11 hydroxylase fusion protein comprises a portion of a C11 hydroxylase protein and a signal peptide from another protein or a synthetic signal peptide. In some embodiments, a C11 hydroxylase fusion protein comprises residues 2-28 of wild-type CYP5491 (SEQ ID NO: 208). In some embodiments, a C11 hydroxylase fusion protein comprises residues 3-28 of wild-type CYP5491 (SEQ ID NO: 208). In some embodiments, a C11 hydroxylase fusion protein comprises residues 29-473 of wild-type CYP5491 (SEQ ID NO: 208). In some embodiments, a C11 hydroxylase fusion protein comprises residues 2-473 of wild-type CYP5491 (SEQ ID NO: 208). In some embodiments, a C11 hydroxylase fusion protein comprises residues 3-473 of wild-type CYP5491 (SEQ ID NO: 208).

In some embodiments, a C11 hydroxylase fusion protein comprises a sequence corresponding to residues 2-28 of wild-type CYP5491 (SEQ ID NO: 208) but includes at least one mutation at an amino acid corresponding to residues 2-28 of wild-type CYP5491 (SEQ ID NO: 208). In some embodiments, a C11 hydroxylase fusion protein comprises residues 3-28 of wild-type CYP5491 (SEQ ID NO: 208) but includes at least one mutation at an amino acid corresponding to residues 3-28 of wild-type CYP5491 (SEQ ID NO: 208). In some embodiments, a C11 hydroxylase fusion protein comprises a sequence corresponding to residues 29-473 of wild-type CYP5491 (SEQ ID NO: 208) but includes at least one mutation at an amino acid corresponding to residues 29-473 of wild-type CYP5491 (SEQ ID NO: 208). In some embodiments, a C11 hydroxylase fusion protein comprises a sequence corresponding to residues 2-473 of wild-type CYP5491 (SEQ ID NO: 208) but includes at least one mutation at an amino acid corresponding to residues 29-473 of wild-type CYP5491 (SEQ ID NO: 208). In some embodiments, a C11 hydroxylase fusion protein comprises a sequence corresponding to residues 3-473 of wild-type CYP5491 (SEQ ID NO: 208) but includes at least one mutation at an amino acid corresponding to residues 29-473 of wild-type CYP5491 (SEQ ID NO: 208). In some embodiments, a C11 hydroxylase fusion comprises a sequence corresponding to residues 29-473 of wild-type CYP5491 and includes one mutation at an amino acid corresponding to residues 29-473 of wild-type CYP5491 (SEQ ID NO: 208). In some embodiments, the mutation is an amino acid substitution, deletion, or insertion.

In some instances, a C11 hydroxylase fusion comprises a sequence with a mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 residues relative to wild-type CYP5491 (SEQ ID NO: 208). In some embodiments, the mutation is an amino acid substitution, deletion, or insertion. In some embodiments, the mutation is located in one or more residues selected from residues: 48, 49, 57, 76, 103-107, 109, 110, 112, 113, 118-120, 209-212, 215, 277, 278, 281, 282, 285, 286, 350-355, 376, and/or 457-459 of CYP5491.

### Signal Peptides

As used in this application, a “signal peptide” or “signal sequence” refers to a localization signal that promotes targeting of a protein. For example, in the co-translational pathway in eukaryotes and prokaryotes, a signal-recognition particle (SRP) binds to signal peptides of proteins that are being translated by ribosomes and targets ribosome-nascent polypeptides to SRP receptors that are present on membranes (e.g., plasma membrane or endoplasmic reticulum membrane). In some embodiments, a signal peptide that is recognized by a SRP is referred to as an SRP-dependent signal sequence. In some embodiments, a signal peptide adopts an alpha-helical structure. In some embodiments, a signal peptide is 5-10, 10-20, 20-30, 30-40, 40-50, 50-60, or 60-70 amino acids in length. In some embodiments, a signal sequence is 5-80 amino acids in length. In some embodiments, a signal peptide is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 amino acids in length. In some embodiments, a signal peptide comprises 1-10, 10-20, 20-30, 30-40, 40-50, 50-60, or 60-70 hydrophobic amino acids. In some embodiments, a signal sequence comprises 5-80 hydrophobic amino acids in length. In some embodiments, a signal peptide comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 hydrophobic amino acids. Non-limiting examples of hydrophobic amino acids include alanine (Ala), valine (Val), leucine (Leu), isoleucine (Ile), phenylalanine (Phe), methionine (Met), tyrosine (Tyr) and tryptophan (Trp). In some instances, a signal peptide is an ER localization signal. In some embodiments, a signal peptide also functions as a stop-transfer sequence. In some embodiments, a signal peptide also functions as a membrane-anchor sequence.

As used in this application, an “SRP-dependent protein” refers to a protein that depends on SRP and the SRP receptor for targeting to a membrane. In some instances, the membrane is the endoplasmic reticulum (ER) membrane. Non-limiting examples of SRP-dependent proteins from S. cerevisiae include AIM20, ALG1, ALG5, ANP1, AUR1, BPT1, CAX4, CBR1, CDC50, CHO2, CPT1, CUE4, CWH43, DAP2, DUR3, ERD2, ERG11, ERG24, ERG25, ERG4, ERG5, ERP2, ERP4, ERV41, FET3, FTH1, FTR1, GAA1, GDA1, GET1, GPI13, GPI14, GPI17, GPI19, GPT2, GRX6, GUP1, HRD1, HXT2, HXT3, IFA38, IPT1, KAR2, KRE27, KTR6, LAS21, MAM3, MCH1, MEP1, MEP2, MEP3, MNN11, MSC2, MSC7, NCP1, NDC1, NHX1, NNF2, OCH1, OST4, PEX3, PGA2, PGA3, PHO8, PH088, PHS1, PIN2, PKR1, PMT1, POM33, RBD2, RTN1, RTN2, SMF3, SNA3, SNA4, SNL1, SPC1, SPC2, SRP102, SSH1, STE14, STE6, SUR1, SUR2, TMN3, TMS1, TSC3, TVP15, TYW1, VAN1, VCX1, VMA16, VMA21, VMA3, VPS68, VRG4, VTC1, YAR028W, YBR287W, YBT1, YCF1, YDL121C, YDR307W, YER053C-A, YET1, YET3, YHR045W, YHR138C, YKL063C, YLR050C, YLR413W, YML018C, YMR134W, YMR221C, YNL146W, YOL019W, YOP1, YPL162C, YPR091C, YRO2, ZRC1, and ZRT2. Table 1 depicts the start and end amino acid positions of signal peptides from non-limiting examples of SRP-dependent proteins.

TABLE 1
Non-limiting examples of signal peptides
from SRP-dependent proteins

| | | End | |
|---|---|---|---|
| | | amino | UniProt |
| | Start amino | acid | Accession |
| Name | acid position | position | Number |
| KAR2 | 1 | 42 | P16474 |
| NCP1 | 1 | 7 | P16603 |
| ERP2 | 1 | 25 | P39704 |
| RBD2 | 1 | 16 | Q12270 |
| SNA3 | 1 | 16 | P14359 |
| SPC2 | 1 | 37 | Q04969 |
| NHX1 | 1 | 61 | Q04121 |
| PGA2 | 1 | 22 | P53903 |
| GRX6 | 1 | 29 | Q12438 |
| YLR413W | 1 | 28 | Q06689 |
| YJL062W | 1 | 32 | P40367 |
| MSC2 | 1 | 6 | Q03455 |
| EMC5 | 1 | 6 | P40540 |
| CHO2 | 1 | 55 | P05374 |
| IFA38 | 1 | 19 | P38286 |
| SUR2 | 1 | 8 | P38992 |
| IPT1 | 1 | 24 | P38954 |
| YET3 | 1 | 6 | Q07451 |
| YPL162C | 1 | 13 | Q12042 |
| ERG11 | 1 | 26 | P10614 |
| SRP102 | 1 | 6 | P36057 |
| GUP1 | 1 | 43 | P53154 |
| CBR1 | 1 | 6 | P38626 |
| YHR138C | 1 | 19 | P38841 |

Non-limiting examples of signal peptide sequences are provided in SEQ ID NOs: 209-232. A C11 hydroxylase fusion may comprise a signal peptide sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, to a signal peptide sequence selected from SEQ ID NOs: 209-232 (Table 4), to a signal peptide sequence disclosed in this application, or to a signal peptide sequence listed in Table 1.

A C11 hydroxylase fusion may comprise a signal peptide sequence that comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 amino acid substitutions, deletions, or insertions relative to: a signal peptide sequence selected from SEQ ID NOs: 209-232 (Table 4); a signal peptide sequence listed in Table 1; or any signal peptide sequence disclosed in this application.

A C11 hydroxylase fusion may comprise a signal peptide sequence that comprises no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 amino acid substitutions, deletions, or insertions relative to: a signal peptide sequence selected from SEQ ID NOs: 209-232 (Table 4); a signal peptide sequence listed in Table 1; or any signal peptide sequence disclosed in this application.

In some embodiments, a C11 hydroxylase fusion comprises a signal peptide sequence that comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 amino acid substitutions, deletions, or insertions relative to: a signal peptide sequence selected from SEQ ID NOs: 209-232 (Table 4); a signal peptide sequence listed in Table 1; or any signal peptide sequence disclosed in this application.

In some embodiments, a C11 hydroxylase fusion comprises a signal peptide sequence that comprises 1 amino acid substitution, deletion, or insertion relative to: a signal peptide sequence selected from SEQ ID NOs: 209-232 (Table 4); a signal peptide sequence listed in Table 1; or any signal peptide sequence disclosed in this application.

A C11 hydroxylase or a C11 hydroxylase fusion of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, to a sequence in Table 4, 5, or 7, or to any C11 hydroxylase or C11 hydroxylase fusion disclosed in this application, or to a sequence selected from SEQ ID NOs:113-114, 129-130, or 257-316.

A C11 hydroxylase or a C11 hydroxylase fusion of the present disclosure may comprise one or more point mutations disclosed in Table 3.

In some embodiments, a C11 hydroxylase fusion comprises a signal peptide from ERG11. In some embodiments, a C11 hydroxylase fusion comprises the first 25 amino acid residues from ERG11 and residues 3-473 from wild-type CYP5491. The amino acid sequence of this ERG11 N-terminal-CYP5491 fusion is provided as SEQ ID NO: 280. In some embodiments, a C11 hydroxylase or a C11 hydroxylase fusion includes a T351M point mutation.

In some embodiments, a C11 hydroxylase of the present disclosure is capable of oxidizing mogrol precursors (e.g., cucurbitadienol, 24,25-dihydroxy-cucurbitadienol, and/or 24,25-epoxy-cucurbitadienol (or 24,25 epoxy cucurbitadienol or 24,25-epoxy cucurbitadienol)). In some embodiments, a C11 hydroxylase of the present disclosure catalyzes the formation of mogrol. In some embodiments, a C11 hydroxylase uses cucurbitadienol as a substrate to produce 11-hydroxy-cucurbitadienol. In some embodiments, a C11 hydroxylase uses 24, 25 dihydroxy cucurbitadienol as a substrate to produce mogrol. In some embodiments, a C11 hydroxylase uses 24,25-epoxy cucurbitadienol to produce 11-hydroxy-24,25-epoxycucurbitadienol.

It should be appreciated that activity, such as specific activity, of a C11 hydroxylase can be determined by any means known to one of ordinary skill in the art. In some embodiments, activity (e.g., specific activity) of a C11 hydroxylase may be measured as the concentration of a mogrol precursor produced or mogrol produced per unit of enzyme per unit time. In some embodiments, a C11 hydroxylase of the present disclosure has an activity (e.g., specific activity) of at least 0.0001-0.001 μmol/min/mg, at least 0.001-0.01 μmol/min/mg, at least 0.01-0.1 μmol/min/mg, or at least 0.1-1 μmol/min/mg, including all values in between.

In some embodiments, the activity, such as specific activity, of a C11 hydroxylase is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, at least 100 fold, at least 1000 fold or at least 10000 fold, including all values in between) greater than that of a control C11 hydroxylase. In some embodiments, the control C11 hydroxylase is wild-type CYP5491 (SEQ ID NO: 208).

Without being bound by any particular theory, in some embodiments, the function of CYP5491 may be a rate limiting step in the mogrol biosynthesis pathway, as CYP5491 can produce 11 oxo mogrol (or 11-oxo-mogrol) as a side product. In some embodiments, mutation of one or more of the amino acids around the active site of wild-type CYP5491 may increase the production of mogrol. In some embodiments, mutation of one or more amino acids around the active site of wild-type CYP5491 increases the ratio of mogrol to oxo mogrol.

In some embodiments, a C11 hydroxylase of the present disclosure produces a ratio of mogrol to oxo mogrol of at least 1.1, at least 1.2, at least 1.3, at least 1.4, at least 1.5, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 55, at least 60, at least 65, at least 70, at least 75, at least 80, at least 85, at least 90, at least 95, or at least 100, including all values in between.

In some embodiments, a C11 hydroxylase of the present disclosure produces increased mogrol compared to a control C11 hydroxylase. In some embodiments, the control C11 hydroxylase is wild-type CYP5491 (SEQ ID NO: 208).

In some embodiments, a C11 hydroxylase of the present disclosure produces at least 1.1, at least 1.2, at least 1.3, at least 1.4, at least 1.5, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 55, at least 60, at least 65, at least 70, at least 75, at least 80, at least 85, at least 90, at least 95, or at least 100 times more mogrol compared to a control C11 hydroxylase, including all values in between. In some embodiments, the control C11 hydroxylase is wild-type CYP5491 (SEQ ID NO: 208).

### Cytochrome P450 Reductase Enzymes

Aspects of the present disclosure provide cytochrome P450 reductase enzymes, which may be useful, for example, in the production of mogrol. Cytochrome P450 reductase is also referred to as NADPH:ferrihemoprotein oxidoreductase, NADPH:hemoprotein oxidoreductase, NADPH:P450 oxidoreductase, P450 reductase, POR, CPR, and CYPOR. These reductases can promote C11 hydroxylase activity by catalyzing electron transfer from NADPH to C11 hydroxylase.

Cytochrome P450 reductases of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, to a cytochrome P450 reductase sequence (e.g., nucleic acid or amino acid sequence) in Table 6 or Table 7, or any P450 reductase sequence disclosed in this application or known in the art, or a sequence selected from SEQ ID NOs: 115, 116, 131, 132, 398-399, and 407-408.

In some embodiments, a cytochrome P450 reductase of the present disclosure is capable of promoting oxidation of a mogrol precursor (e.g., cucurbitadienol, 11-hydroxycucurbitadienol, 24,25-dihydroxy-cucurbitadienol, and/or 24,25-epoxy-cucurbitadienol). In some embodiments, a cytochrome P450 reductase of the present disclosure catalyzes the formation of a mogrol precursor or mogrol.

It should be appreciated that activity, such as specific activity, of a cytochrome P450 reductase can be measured by any means known to one of ordinary skill in the art. In some embodiments, such activity may be measured as the concentration of a mogrol precursor produced or mogrol produced per unit enzyme per unit time in the presence of a C11 hydroxylase. In some embodiments, a cytochrome P450 reductase of the present disclosure has an activity, such as specific activity, of at least 0.0001-0.001 mol/min/mg, at least 0.001-0.01 mol/min/mg, at least 0.01-0.1 mol/min/mg, or at least 0.1-1 mol/min/mg, including all values in between.

In some embodiments, the activity, such as specific activity, of a cytochrome P450 reductase is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, at least 100 fold, at least 1000 fold or at least 10000 fold, including all values in between) greater than that of a control cytochrome P450 reductase.

### Epoxide Hydrolase Enzymes (EPHs)

Aspects of the present disclosure provide epoxide hydrolase enzymes (EPHs), which may be useful, for example, in the conversion of 24-25 epoxy-cucurbitadienol to 24-25 dihydroxy-cucurbitadienol or in the conversion of 11-hydroxy-24,25-epoxycucurbitadienol to mogrol. EPHs are capable of converting an epoxide into two hydroxyls.

EPHs of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, to an EPH sequence (e.g., nucleic acid or amino acid sequence) in Table 6 or Table 7, or to any of the EPH sequences disclosed in this application or known in the art, or to a sequence selected from SEQ ID NOs: 117-125, 133-141, 401-402, and 410-411.

In some embodiments, a recombinant EPH of the present disclosure is capable of promoting hydrolysis of an epoxide in a cucurbitadienol compound (e.g., hydrolysis of the epoxide in 24-25 epoxy-cucurbitadienol). In some embodiments, an EPH of the present disclosure catalyzes the formation of a mogrol precursor (e.g., 24-25 dihydroxy-cucurbitadienol).

It should be appreciated that activity, such as specific activity, of an EPH can be measured by any means known to one of ordinary skill in the art. In some embodiments, activity, such as specific activity, of an EPH may be measured as the concentration of a mogrol precursor (e.g., 24-25 dihydroxy-cucurbitadienol) or mogrol produced. In some embodiments, the concentration of mogrol precursor, including 24,25 dihydroxy-cucurbitadienol, is measured in terms of, for example, normalized peak areas of a chromatogram, rather than absolute titer. In some embodiments, use of normalized peak areas is useful when there is a lack of an analytical standard for a mogrol precursor.

### Squalene Epoxidase Enzymes (SQEs)

Aspects of the present disclosure provide SQEs, which are capable of oxidizing a squalene (e.g., squalene or 2-3-oxidosqualene) to produce a squalene epoxide (e.g., 2-3-oxidosqualene or 2-3, 22-23-diepoxysqualene). SQEs may also be referred to as squalene monooxygenases.

SQEs of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, to an SQE sequence (e.g., nucleic acid or amino acid sequence) in Table 6 or Table 7, or to any of the SQEs disclosed in this application or known in the art, or to a sequence selected from SEQ ID NOs: 126-128, 142-144, 404, or 413.

In some embodiments, a recombinant SQE of the present disclosure is capable of promoting formation of an epoxide in a squalene compound (e.g., epoxidation of squalene or 2,3-oxidosqualene). In some embodiments, an SQE of the present disclosure catalyzes the formation of a mogrol precursor (e.g., 2-3-oxidosqualene or 2-3, 22-23-diepoxysqualene) or 24,25 diepoxy-cucurbitadienol).

Activity of a recombinant SQE may be measured by determining the increase in levels of 2-3, 22-23 diepoxy cucurbitadienol and/or dihydroxycucurbitadienol by normalized chromatogram peak area improvement over a control enzyme. In some embodiments, the control enzyme is ERG1 (SEQ ID NO: 413) in Table 6. In some embodiments, activity, such as specific activity, of a recombinant SQE may be measured as the concentration of a mogrol precursor (e.g., 2-3-oxidosqualene or 2-3, 22-23-diepoxysqualene or 24,25-diepoxy-cucurbitadienol) produced per unit of enzyme per unit of time.

Without being bound by a particular theory, expression of an SQE in a host cell may increase the production of a mogrol precursor (e.g., epoxy-cucurbitadineol, squalene-dioxide and 2,3-oxidosqualene).

In some embodiments, potential toxicity associated with expression of an SQE is mitigated. As a non-limiting example, methods of reducing toxicity can include increasing expression of downstream enzymes, including EPH and/or C11 hydroxylase with a cytochrome P450 reductase. Without being bound by a particular theory, expression of EPH and/or C11 hydroxylase with a cytochrome P450 reductase may reduce toxicity by promoting the conversion of epoxide molecules into either dihydroxy cucurbitadienol or mogrol. In some embodiments, toxicity reduction comprises expressing one or more UGTs. Without being bound by a particular theory, expression of one or more UGTs may reduce toxicity by increasing the production of glycosylated compounds.

### Cucurbitadienol Synthase (CDS) Enzymes

Aspects of the present disclosure provide cucurbitadienol synthase (CDS) enzymes, which may be useful, for example, in the production of a cucurbitadienol compound, such as 24-25 epoxy-cucurbitadienol or cucurbitadienol). CDSs are capable of catalyzing the formation of cucurbitadienol compounds, such as 24-25 epoxy-cucurbitadienol or cucurbitadienol from oxidosqualene (e.g., 2-3-oxidosqualene or 2,3; 22,23-diepoxysqualene).

In some embodiments, CDSs have a leucine at a residue corresponding to position 123 of SEQ ID NO: 74 that distinguishes them from other oxidosqualene cyclases, as discussed in Takase et al. Org. Biomol. Chem., 2015, 13, 7331-7336, which is incorporated by reference in its entirety.

CDSs of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical, including all values in between, to a nucleic acid or amino acid sequence in Table 8, to a sequence selected from SEQ ID NOs: 1-80, or to any other CDS sequence disclosed in this application or known in the art.

In some embodiments a CDS enzyme corresponds to AquAagaCDS16 (SEQ ID NO: 43), CSPI06G07180.1 (SEQ ID NO: 52), or A0A1S3CBF6 (SEQ ID NO: 49).

In some embodiments, a nucleic acid sequence encoding a CDS enzyme may be re-coded for expression in a particular host cell, including S. cerevisiae. In some embodiments, a re-coded nucleic acid sequence encoding a CDS enzyme corresponds to SEQ ID NO: 34.

In some embodiments, a CDS of the present disclosure is capable of using oxidosqualene (e.g., 2,3-oxidosqualene or 2,3; 22,23-diepoxysqualene) as a substrate. In some embodiments, a CDS of the present disclosure is capable of producing cucurbitadienol compounds (e.g., 24-25 epoxy-cucurbitadienol or cucurbitadienol). In some embodiments, a CDS of the present disclosure catalyzes the formation of cucurbitadienol compounds (e.g., 24-25 epoxy-cucurbitadienol or cucurbitadienol) from oxidosqualene (e.g., 2-3-oxidosqualene or 2,3; 22,23-diepoxysqualene).

It should be appreciated that activity of a CDS can be measured by any means known to one of ordinary skill in the art. In some embodiments, the activity of a CDS may be measured as the normalized peak area of cucurbitadienol produced. In some embodiments, this activity is measured in arbitrary units. In some embodiments, the activity, such as specific activity, of a CDS of the present disclosure is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, or at least 100 fold, including all values in between) greater than that of a control CDS.

It should be appreciated that one of ordinary skill in the art would be able to characterize a protein as a CDS enzyme based on structural and/or functional information associated with the protein. For example, in some embodiments, a protein can be characterized as a CDS enzyme based on its function, such as the ability to produce cucurbitadienol compounds (e.g., 24-25 epoxy-cucurbitadienol or cucurbitadienol) using oxidosqualene (e.g., 2,3-oxidosqualene or 2,3; 22,23-diepoxysqualene) as a substrate. In some embodiments, a protein can be characterized, at least in part, as a CDS enzyme based on the presence of a leucine residue at a position corresponding to position 123 of SEQ ID NO: 74.

In some embodiments, a recombinant host cell that expresses a heterologous gene encoding a cucurbitadienol synthase (CDS) enzyme produces at least 10%, 20%, 30%, 40%, 50%, 60%, 70%, 80%, 90%, or 100% more cucurbitadienol compound compared to the same recombinant host cell that does not express the heterologous gene.

In other embodiments, a protein can be characterized as a CDS enzyme based on the percent identity between the protein and a known CDS enzyme. For example, the protein may be at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical, including all values in between, to any of the CDS sequences described in this application or the sequence of any other CDS enzyme. In other embodiments, a protein can be characterized as a CDS enzyme based on the presence of one or more domains in the protein that are associated with CDS enzymes. For example, in certain embodiments, a protein is characterized as a CDS enzyme based on the presence of a substrate channel and/or an active-site cavity characteristic of CDS enzymes known in the art. In some embodiments, the active-site cavity comprises a residue that acts a gate to this channel, helping to exclude water from the cavity. In some embodiments, the active-site comprises a residue that acts a proton donor to open the epoxide of the substrate and catalyze the cyclization process.

In other embodiments, a protein can be characterized as a CDS enzyme based on a comparison of the three-dimensional structure of the protein compared to the three-dimensional structure of a known CDS enzyme. It should be appreciated that a CDS enzyme can be a synthetic protein.

### UDP-Glycosyltransferases (UGT) Enzymes

Aspects of the present disclosure provide UDP-glycosyltransferase enzymes (UGTs), which may be useful, for example, in the production of a mogroside (e.g., mogroside I-A1 (MIA1), mogroside I-E (MIE), mogroside II-A1 (MIIA1), mogroside II-A2 (MIIA2), mogroside III-A1 (MIIIA1), mogroside II-E (MIIE), mogroside III (MIII), siamenoside I, mogroside III-E (MIIIE), mogroside IV, mogroside IVa, isomogroside IV, mogroside V, or mogroside VI).

As used in this application, a UGT is an enzyme that is capable of catalyzing the addition of the glycosyl group from a UTP-sugar to a compound (e.g., mogroside or mogrol). Structurally, UGTs often comprise a UDPGT (Prosite: PS00375) domain and a catalytic dyad. As a non-limiting example, one of ordinary skill in the art may identify a catalytic dyad in a UGT by aligning the UGT sequence to UGT94-289-1 (a wildtype UGT sequence from the monk fruit Siraitia grosvenorii) and identifying the two residues in the UGT that correspond to histidine 21 (H21) and aspartate 122 (D122) of UGT94-289-1.

The amino acid sequence for UGT94-289-1 is:

(SEQ ID NO: 109)
MDAQRGHTTTILMFPWLGYGHLSAFLELAKSLSRRNFHIYFCSTSVNLDA
IKPKLPSSSSSDSIQLVELCLPSSPDQLPPHLHTTNALPPHLMPTLHQAF
SMAAQHFAAILHTLAPHLLIYDSFQPWAPQLASSLNIPAINFNTTGASVL
TRMLHATHYPSSKFPISEFVLHDYWKAMYSAAGGAVTKKDHKIGETLANC
LHASCSVILINSFRELEEKYMDYLSVLLNKKVVPVGPLVYEPNQDGEDEG
YSSIKNWLDKKEPSSTVFVSFGSEYFPSKEEMEEIAHGLEASEVHFIWVV
RFPQGDNTSAIEDALPKGFLERVGERGMVVKGWAPQAKILKHWSTGGFVS
HCGWNSVMESMMFGVPIIGVPMHLDQPFNAGLAEEAGVGVEAKRDPDGKI
QRDEVAKLIKEVVVEKTREDVRKKAREMSEILRSKGEEKMDEMVAAISLF
LKI.

A non-limiting example of a nucleic acid sequence encoding UGT94-289-1 is:

(SEQ ID NO: 93)
ATGGACGCGCAACGCGGACATACGACTACCATCCTGATGTTTCCGTGGTT
GGGGTACGGCCACCTTAGTGCATTCCTCGAATTAGCCAAGAGCTTGTCGC
GTAGGAACTTTCATATTTATTTCTGTTCCACATCTGTCAATTTAGATGCT
ATAAAACCCAAACTACCATCATCTTCAAGTTCCGATTCTATTCAGCTTGT
AGAGTTATGCTTGCCTTCCTCGCCAGACCAACTACCCCCACACCTGCATA
CAACTAATGCTCTACCTCCACATCTAATGCCTACCCTGCACCAGGCCTTT
TCAATGGCAGCTCAACATTTTGCAGCTATATTACATACTTTAGCACCGCA
CTTGTTAATCTATGATTCGTTCCAGCCTTGGGCGCCACAATTGGCCAGCT
CTCTTAACATTCCTGCTATTAATTTTAATACCACGGGTGCCAGTGTGCTA
ACAAGAATGTTACACGCGACTCATTACCCATCTTCAAAGTTCCCAATCTC
CGAATTTGTTTTACATGATTATTGGAAAGCAATGTATTCAGCAGCTGGTG
GTGCTGTTACAAAAAAGGACCATAAAATAGGAGAAACCTTGGCAAACTGT
TTACACGCTTCTTGCTCGGTAATTCTGATCAATTCATTCAGAGAGTTGGA
AGAAAAATACATGGATTACTTGTCTGTCTTACTAAACAAGAAAGTTGTGC
CCGTGGGTCCGCTTGTTTATGAGCCAAACCAAGATGGCGAAGACGAAGGT
TATAGTTCGATAAAGAATTGGCTCGATAAAAAGGAGCCCTCCTCAACTGT
CTTTGTTTCCTTCGGGTCCGAATATTTTCCGTCCAAAGAAGAAATGGAAG
AAATTGCCCATGGCTTGGAGGCTAGCGAGGTACACTTTATTTGGGTCGTT
AGATTCCCACAAGGAGACAATACTTCTGCAATTGAAGATGCCCTTCCTAA
GGGTTTTCTTGAGCGAGTGGGCGAACGTGGAATGGTGGTTAAGGGTTGGG
CTCCTCAGGCCAAAATTTTGAAACATTGGAGCACAGGCGGTTTCGTAAGT
CATTGTGGATGGAATAGTGTTATGGAGAGCATGATGTTTGGTGTACCCAT
AATAGGTGTTCCGATGCATTTAGATCAACCATTTAATGCAGGGCTCGCGG
AAGAAGCAGGAGTAGGGGTAGAGGCTAAAAGGGACCCTGATGGTAAGATA
CAGAGAGATGAAGTCGCTAAACTGATCAAAGAAGTGGTTGTCGAAAAAAC
GCGCGAAGATGTCAGAAAGAAGGCTAGGGAAATGTCTGAAATTTTACGTT
CGAAAGGTGAGGAAAAGATGGACGAGATGGTTGCAGCCATTAGTCTCTTC
TTGAAGATATAA.

One of ordinary skill in the art would readily recognize how to determine for any UGT enzyme what amino acid residue corresponds to a specific amino acid residue in UGT94-289-1 (SEQ ID NO: 109) by, for example, aligning sequences and/or by comparing secondary structures.

In some embodiments, a UGT of the present disclosure comprises a sequence (e.g., nucleic acid or amino acid sequence) that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical, including all values in between, to a sequence in Table 9, or to a sequence selected from SEQ ID NOs: 81-112, or to any UGT sequence disclosed in this application or known in the art.

In some embodiments, a UGT of the present disclosure may comprise an amino acid substitution at an amino acid residue corresponding to an amino acid residue in wild-type UGT94-289-1 (SEQ ID NO: 109). A UGT of the present disclosure can comprise a conservative amino acid substitution and/or a non-conservative amino acid substitution. In some embodiments, a UGT of the present disclosure comprises 1, 2, 3, 4, 5, 6, 7, 8, 9, 10 or more than 10 conservative amino acid substitution(s). In some embodiments, a UGT of the present disclosure comprises 1, 2, 3, 4, 5, 6, 7, 8, 9, 10 or more than 10 non-conservative amino acid substitutions. In some embodiments, a conservative or non-conservative amino acid substitution is not located in a conserved region of a UGT protein. In some embodiments, a conservative or non-conservative amino acid substitution is not located in a region corresponding to: residues 83 to 92; residues 179 to 198; residue N143; residue L374; residue H21; or residue D122 of wild-type UGT94-289-1. One of ordinary skill in the art would readily be able to test a UGT that comprises a conservative and/or non-conservative substitution to determine whether the conservative and/or non-conservative substitution impacts the activity or function of the UGT.

An amino acid residue that contains a substitution can be an amino acid that corresponds to an amino acid residue in wild-type UGT94-289-1 (SEQ ID NO: 109) selected from, e.g., S123; F124; N143; T144; T145; V149; Y179; G18; S180; A181; G184; A185; V186; T187; K189; Y19; H191; K192; G194; E195; A198; F276; N355; H373; L374; N47; H83; T84; T85; N86; P89; and/or L92. Non-limiting examples of such amino acid substitutions include: S123 may be mutated to alanine, cysteine, glycine or valine, or to any conservative substitution of alanine, cysteine, glycine or valine; F124 may be mutated to tyrosine or to any conservative substitution of tyrosine; N143 may be mutated to alanine, cysteine, glutamate, isoleucine, leucine, methionine, glutamine, serine, threonine or valine, or to any conservative substitution of alanine, cysteine, glutamate, isoleucine, leucine, methionine, glutamine, serine, threonine or valine; T144 may be mutated to alanine, cysteine, asparagine or proline, or to any conservative substitution of alanine, cysteine, asparagine or proline; T145 may be mutated to alanine, cysteine, glycine, methionine, asparagine, glutamine, or serine, or any conservative substitution of alanine, cysteine, glycine, methionine, asparagine, glutamine, or serine; V149 may be mutated to cysteine, leucine or methionine, or to any conservative substitution of cysteine, leucine or methionine; Y179 may be mutated to glutamate, phenylalanine, histidine, isoleucine, lysine, leucine, valine, or tryptophan, or to any conservative substitution glutamate, phenylalanine, histidine, isoleucine, lysine, leucine, valine, or tryptophan; G18 may be mutated to serine or to any conservative substitution of serine; S180 may be mutated to alanine or valine, or to any conservative substitution of alanine or valine; A181 may be mutated to lysine or threonine, or to any conservative substitution of lysine or threonine; G184 may be mutated to alanine, cysteine, aspartate, glutamate, phenylalanine, histidine, isoleucine, lysine, methionine, asparagine, proline, glutamine, arginine, serine, threonine, or tyrosine, or to any conservative substitution of alanine, cysteine, aspartate, glutamate, phenylalanine, histidine, isoleucine, lysine, methionine, asparagine, proline, glutamine, arginine, serine, threonine, or tyrosine; A185 may be mutated to cysteine, aspartate, glutamate, glycine, lysine, leucine, methionine, asparagine, proline, glutamine, threonine, tryptophan or tyrosine, or to any conservative substitution of cysteine, aspartate, glutamate, glycine, lysine, leucine, methionine, asparagine, proline, glutamine, threonine, tryptophan or tyrosine; V186 may be mutated to alanine, cysteine, aspartate, glutamate, glycine, isoleucine, lysine, leucine, methionine, asparagine, proline, glutamine, arginine, threonine, tryptophan, or tyrosine, or to any conservative substitution of alanine, cysteine, aspartate, glutamate, glycine, isoleucine, lysine, leucine, methionine, asparagine, proline, glutamine, arginine, threonine, tryptophan, or tyrosine; T187 may be mutated to alanine, cysteine, aspartate, glutamate, glycine, histidine, isoleucine, lysine, leucine, asparagine, proline, arginine, serine, valine, tryptophan, or tyrosine, or to any conservative substitution of alanine, cysteine, aspartate, glutamate, glycine, histidine, isoleucine, lysine, leucine, asparagine, proline, arginine, serine, valine, tryptophan, or tyrosine; K189 may be mutated to alanine, cysteine, aspartate, glutamate, phenylalanine, glycine, histidine, isoleucine, leucine, methionine, proline, glutamine, arginine, serine, threonine, valine, tryptophan, or tyrosine, or to any conservative substitution thereof of alanine, cysteine, aspartate, glutamate, phenylalanine, glycine, histidine, isoleucine, leucine, methionine, proline, glutamine, arginine, serine, threonine, valine, tryptophan, or tyrosine; Y19 may be mutated to phenylalanine, histidine, leucine, or valine, or to any conservative substitution of phenylalanine, histidine, leucine, or valine; H191 may be mutated to alanine, cysteine, aspartate, glutamate, glycine, lysine, methionine, proline, glutamine, serine, threonine, valine, tryptophan, or tyrosine, or to any conservative substitution of mutated to alanine, cysteine, aspartate, glutamate, glycine, lysine, methionine, proline, glutamine, serine, threonine, valine, tryptophan, or tyrosine; K192 may be mutated to cysteine or phenylalanine, or to any conservative substitution of cysteine or phenylalanine; G194 may be mutated to aspartate, leucine, methionine, asparagine, proline, serine, or tryptophan, or to any conservative substitution of aspartate, leucine, methionine, asparagine, proline, serine, or tryptophan; E195 may be mutated to alanine, isoleucine, lysine, leucine, asparagine, glutamine, serine, threonine, or tyrosine, or to any conservative substitution of alanine, isoleucine, lysine, leucine, asparagine, glutamine, serine, threonine, or tyrosine; A198 may be mutated to cysteine, aspartate, glutamate, phenylalanine, histidine, isoleucine, lysine, leucine, methionine, asparagine, proline, glutamine, arginine, serine, threonine, valine, or tyrosine, or to any conservative substitution of cysteine, aspartate, glutamate, phenylalanine, histidine, isoleucine, lysine, leucine, methionine, asparagine, proline, glutamine, arginine, serine, threonine, valine, or tyrosine; F276 may be mutated to cysteine or glutamine, or to any conservative substitution of cysteine or glutamine; N355 may be mutated to glutamine or serine, or any conservative substitution thereof; H373 may be mutated to lysine, leucine, methionine, arginine, valine, or tyrosine, or to any conservative substitution of lysine, leucine, methionine, arginine, valine, or tyrosine; L374 may be mutated to alanine, cysteine, phenylalanine, histidine, methionine, asparagine, glutamine, serine, threonine, valine, tryptophan, or tyrosine, or to any conservative substitution of alanine, cysteine, phenylalanine, histidine, methionine, asparagine, glutamine, serine, threonine, valine, tryptophan, or tyrosine; N47 may be mutated to glycine or to any conservative substitution of glycine; H83 may be mutated to glutamine or tryptophan, or to any conservative substitution of glutamine or tryptophan; T84 may be mutated to tyrosine or to any conservative substitution of tyrosine; T85 may be mutated to glycine, lysine, proline, serine, or tyrosine, or to any conservative substitution of glycine, lysine, proline, serine, or tyrosine; N86 may be mutated to alanine, cysteine, glutamate, isoleucine, lysine, leucine, serine, tryptophan, or tyrosine, or to any conservative substitution of alanine, cysteine, glutamate, isoleucine, lysine, leucine, serine, tryptophan, or tyrosine; P89 may be mutated to methionine or serine or to any conservative substitution of methionine or serine; and/or L92 may be mutated to histidine or lysine or to any conservative substitution of histidine or lysine.

In some embodiments, a UGT enzyme contains an amino acid substitution located within 10 angstrom, 9 angstrom 8 angstrom, 7 angstrom, 6 angstrom, 5 angstrom, 4 angstrom, 3 angstrom, 2 angstrom, or within 1 angstrom (including all values in between) of a catalytic dyad. The catalytic dyad may correspond to residues 21 and 122 of wildtype UGT94-289-1 (e.g., histidine 21 and aspartate acid 122). It should be appreciated that one of ordinary skill in the art would readily recognize how to determine to corresponding location of the catalytic dyad in any UGT enzyme, for example, by aligning the sequence and/or by comparing the secondary structure with UGT94-289-1 (SEQ ID NO: 109).

In some embodiments, a UGT enzyme contains an amino acid substitution at an amino acid residue located in one or more structural motifs of the UGT. Non-limiting examples of secondary structures in UGTs, such as UGT94-289-1 (SEQ ID NO: 109), include: the loop between beta sheet 4 and alpha helix 5; beta sheet 5; the loop between beta sheet 5 and alpha helix 6; alpha helix 6; the loop between alpha helix 6 & 7; the loop between beta sheet 1 & alpha helix 1; alpha helix 7; the loop between alpha helix 7 & 8; alpha helix 1; alpha helix 8; the loop between beta sheet 8 & alpha helix 13; alpha helix 17; the loop between beta sheet 12 & alpha helix 18; alpha helix 2; loop between beta sheet 3 & alpha helix 3; alpha helix 3; and the loop between alpha helix 3 & 4; loop 8; beta sheet 5; loop 10; alpha helix 5; loop 11; loop 2; alpha helix 6; loop 12; alpha helix 1; alpha helix 7; loop 18; alpha helix 14; loop 26; alpha helix 2; loop 6; and alpha helix 3.

In some embodiments, the UGT comprises an amino acid substitution at an amino acid residue corresponding to the amino acid residue in wild-type UGT94-289-1 (SEQ ID NO: 109) selected from: N143 and L374. In some embodiments, the residue corresponding to N143 is mutated to a negatively charged R group, a polar uncharged R group, or a nonpolar aliphatic R group. In some embodiments, the residue corresponding to L374 is mutated to a nonpolar aliphatic R group, a positively charged R group, a polar uncharged R group, or a nonpolar aromatic R group.

The UGTs of the present disclosure may be capable of glycosylating mogrol or a mogroside at any of the oxygenated sites (e.g., at C3, C11, C24, and C25). In some embodiments, the UGT is capable of branching glycosylation (e.g., branching glycosylation of a mogroside at C3 or C24).

Non-limiting examples of suitable substrates for the UGTs of the present disclosure include mogrol and mogrosides (e.g., mogroside IA1 (MIA1), mogroside IE (MIE), mogroside II-A1 (MIIA1), mogroside III-A1 (MIIIA1), mogroside II-E (MIIE), mogroside III (MIII), or mogroside III-E (MIIIE), siamenoside I).

In some embodiments, the UGTs of the present disclosure are capable of producing mogroside IA1 (MIA1), mogroside IE (MIE), mogroside II-A1 (MIIA1), mogroside II-A2 (MIIA2), mogroside III-A1 (MIIIA1), mogroside II-E (MIIE), mogroside III (MIII), siamenoside I, mogroside III-E (MIIIE), mogroside IV, mogroside IVa, isomogroside IV, and/or mogroside V.

In some embodiments, the UGT is capable of catalyzing the conversion of mogrol to MIA1; mogrol to MIE1; MIA1 to MIIA1; MIE1 to MIIE; MIIA1 to MIIIA1; MIA1 to MIIE; MIIA1 to MIII; MIIIA1 to siamenoside I; MIIE to MIII; MIII to siamenoside I; MIIE to MIIE; and/or MIIIE to siamenoside I.

It should be appreciated that activity, such as specific activity, of a UGT can be measured by any means known to one of ordinary skill in the art. In some embodiments, the activity, such as specific activity, of a UGT (e.g., a variant UGT) may be determined by measuring the amount of glycosylated mogroside produced per unit enzyme per unit time. For example, the activity, such as specific activity, may be measured in mmol glycosylated mogroside target produced per gram of enzyme per hour. In some embodiments, a UGT of the present disclosure (e.g., variant UGT) may have an activity, such as specific activity, of at least 0.1 mmol (e.g., at least 1 mmol, at least 1.5 mmol, at least 2 mmol, at least 2.5 mmol, at least 3, at least 3.5 mmol, at least 4 mmol, at least 4.5 mmol, at least 5 mmol, at least 10 mmol, including all values in between) glycosylated mogroside target produced per gram of enzyme per hour.

In some embodiments, the activity, such as specific activity, of a UGT of the present disclosure is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, or at least 100 fold, including all values in between) greater than that of a control UGT. In some embodiments, the control UGT is UGT94-289-1 (SEQ ID NO: 109). In some embodiments, for a UGT that has an amino acid substitution, a control UGT is the same UGT but without the amino acid substitution.

It should be appreciated that one of ordinary skill in the art would be able to characterize a protein as a UGT enzyme based on structural and/or functional information associated with the protein. For example, a protein can be characterized as a UGT enzyme based on its function, such as the ability to produce one or more mogrosides in the presence of a mogroside precursor, such as mogrol.

In other embodiments, a protein can be characterized as a UGT enzyme based on the percent identity between the protein and a known UGT enzyme. For example, the protein may be at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical, including all values in between, to any of the UGT sequences described in this application or the sequence of any other UGT enzyme. In other embodiments, a protein can be characterized as a UGT enzyme based on the presence of one or more domains in the protein that are associated with UGT enzymes. For example, in certain embodiments, a protein is characterized as a UGT enzyme based on the presence of a sugar binding domain and/or a catalytic domain, characteristic of UGT enzymes known in the art. In certain embodiments, the catalytic domain binds the substrate to be glycosylated.

In other embodiments, a protein can be characterized as a UGT enzyme based on a comparison of the three-dimensional structure of the protein compared to the three-dimensional structure of a known UGT enzyme. For example, a protein could be characterized as a UGT based on the number or position of alpha helical domains, beta-sheet domains, etc. It should be appreciated that a UGT enzyme can be a synthetic protein.

In some embodiments, the UGT does not comprise the sequence of SEQ ID NO: 109. In some embodiments, a UGT comprises less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 79%, less than 78%, less than 77%, less than 76%, less than 75%, less than 74%, less than 73%, less than 72%, less than 71%, or less than 70% identity to SEQ ID NO: 109.

### Variants

Aspects of the disclosure relate to nucleic acids encoding any of the recombinant polypeptides described, such as CDS, UGT, C11 hydroxylase, cytochrome P450 reductase, EPH, SQE, CDS, and UGT enzymes. Variants of nucleic acid or amino acid sequences described in this application are also encompassed by the present disclosure. A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between.

Unless otherwise noted, the term “sequence identity,” as known in the art, refers to a relationship between the sequences of two polypeptides or polynucleotides, as determined by sequence comparison (alignment). In some embodiments, sequence identity is determined across the entire length of a sequence, such as a reference sequence, while in other embodiments, sequence identity is determined over a region of a sequence. In some embodiments, sequence identity is determined over a region (e.g., a stretch of amino acids or nucleic acids, e.g., the sequence spanning an active site) of a sequence (e.g., C11 hydroxylase, cytochrome P450 reductase, EPH, SQE, UGT, or CDS sequence). For example, in some embodiments, sequence identity is determined over a region corresponding to at least 30%, at least 40%, at least 50%, at least 60%, at least 70%, at least 80%, at least 90%, at least 95%, or over 100% of the length of the reference sequence.

Identity measures the percent of identical matches between the smaller of two or more sequences with gap alignments (if any) addressed by a particular mathematical model, algorithm, or computer program.

Identity of related polypeptides or nucleic acid sequences can be readily calculated by any of the methods known to one of ordinary skill in the art. The percent identity of two sequences (e.g., nucleic acid or amino acid sequences) may, for example, be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3 to obtain amino acid sequences homologous to the proteins described in this application. Where gaps exist between two sequences, Gapped BLAST® can be utilized, for example, as described in Altschul et al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences. In some embodiments, the identity of two nucleic acids is determined by aligning the two nucleotide sequences and calculating the number of identical nucleotide and dividing by the length of one of the nucleic acids.

For multiple sequence alignments, computer programs including Clustal Omega (Sievers et al., Mol Syst Biol. 2011 Oct. 11; 7:539) may be used.

In preferred embodiments, a sequence, including a nucleic acid or amino acid sequence, is found to have a specified percent identity to a reference sequence, such as a sequence disclosed in this application and/or recited in the claims when sequence identity is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993 (e.g., BLAST®, NBLAST®, XBLAST® or Gapped BLAST® programs, using default parameters of the respective programs).

In some embodiments, a sequence, including a nucleic acid or amino acid sequence, is found to have a specified percent identity to a reference sequence, such as a sequence disclosed in this application and/or recited in the claims when sequence identity is determined using the Smith-Waterman algorithm (Smith, T. F. & Waterman, M. S. (1981) “Identification of common molecular subsequences.” J. Mol. Biol. 147:195-197) or the Needleman-Wunsch algorithm (Needleman, S. B. & Wunsch, C. D. (1970) “A general method applicable to the search for similarities in the amino acid sequences of two proteins.” J. Mol. Biol. 48:443-453) using default parameters.

In some embodiments, a sequence, including a nucleic acid or amino acid sequence, is found to have a specified percent identity to a reference sequence, such as a sequence disclosed in this application and/or recited in the claims when sequence identity is determined using a Fast Optimal Global Sequence Alignment Algorithm (FOGSAA) using default parameters.

In some embodiments, a sequence, including a nucleic acid or amino acid sequence, is found to have a specified percent identity to a reference sequence, such as a sequence disclosed in this application and/or recited in the claims when sequence identity is determined using Clustal Omega (Sievers et al., Mol Syst Biol. 2011 Oct. 11; 7:539) using default parameters.

As used in this application, a residue (such as a nucleic acid residue or an amino acid residue) in sequence “X” is referred to as corresponding to a position or residue (such as a nucleic acid residue or an amino acid residue) “n” in a different sequence “Y” when the residue in sequence “X” is at the counterpart position of “n” in sequence “Y” when sequences X and Y are aligned using amino acid sequence alignment tools known in the art.

Variant sequences may be homologous sequences. As used in this application, homologous sequences are sequences (e.g., nucleic acid or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity, including all values in between). Homologous sequences include but are not limited to paralogous or orthologous sequences. Paralogous sequences arise from duplication of a gene within a genome of a species, while orthologous sequences diverge after a speciation event.

In some embodiments, a polypeptide variant (e.g., C11 hydroxylase, cytochrome P450 reductase, EPH, SQE, UGT, or CDS variant) comprises a domain that shares a secondary structure (e.g., alpha helix, beta sheet) with a reference polypeptide (e.g., a reference C11 hydroxylase, cytochrome P450 reductase, EPH, SQE, CDS, or UGT). In some embodiments, a polypeptide variant (e.g., C11 hydroxylase, cytochrome P450 reductase, EPH, SQE, CDS, or UGT variant) shares a tertiary structure with a reference polypeptide (e.g., a reference C11 hydroxylase, cytochrome P450 reductase, EPH, SQE, CDS, or UGT). As a non-limiting example, a variant polypeptide may have low primary sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity) compared to a reference polypeptide, but share one or more secondary structures (e.g., including but not limited to loops, alpha helices, or beta sheets, or have the same tertiary structure as a reference polypeptide. For example, a loop may be located between a beta sheet and an alpha helix, between two alpha helices, or between two beta sheets. Homology modeling may be used to compare two or more tertiary structures.

Mutations can be made in a nucleotide sequence by a variety of methods known to one of ordinary skill in the art. For example, mutations can be made by PCR-directed mutation, site-directed mutagenesis according to the method of Kunkel (Kunkel, Proc. Nat. Acad. Sci. U.S.A. 82: 488-492, 1985), by chemical synthesis of a gene encoding a polypeptide, by gene editing tools such as CRISPR, or by insertions, such as insertion of a tag (e.g., a HIS tag or a GFP tag). Mutations can include, for example, substitutions, deletions, and translocations, generated by any method known in the art. Methods for producing mutations may be found in references such as Molecular Cloning: A Laboratory Manual, J. Sambrook, et al., eds., Fourth Edition, Cold Spring Harbor Laboratory Press, Cold Spring Harbor, N.Y., 2012, or Current Protocols in Molecular Biology, F. M. Ausubel, et al., eds., John Wiley & Sons, Inc., New York, 2010.

In some embodiments, methods for producing variants include circular permutation (Yu and Lutz, Trends Biotechnol. 2011 Jan.; 29(1):18-25. In circular permutation, the linear primary sequence of a polypeptide can be circularized (e.g., by joining the N-terminal and C-terminal ends of the sequence) and the polypeptide can be severed (“broken”) at a different location. Thus, the linear primary sequence of the new polypeptide may have low sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less or less than 5%, including all values in between) as determined by linear sequence alignment methods (e.g., Clustal Omega or BLAST). Topological analysis of the two proteins, however, may reveal that the tertiary structure of the two polypeptides is similar or dissimilar. Without being bound by a particular theory, a variant polypeptide created through circular permutation of a reference polypeptide and with a similar tertiary structure as the reference polypeptide can share similar functional characteristics (e.g., enzymatic activity, enzyme kinetics, substrate specificity or product specificity). In some instances, circular permutation may alter the secondary structure, tertiary structure or quaternary structure and produce an enzyme with different functional characteristics (e.g., increased or decreased enzymatic activity, different substrate specificity, or different product specificity). See, e.g., Yu and Lutz, Trends Biotechnol. 2011 January; 29(1):18-25.

It should be appreciated that in a protein that has undergone circular permutation, the linear amino acid sequence of the protein would differ from a reference protein that has not undergone circular permutation. However, one of ordinary skill in the art would be able to readily determine which residues in the protein that has undergone circular permutation correspond to residues in the reference protein that has not undergone circular permutation by, for example, aligning the sequences and detecting conserved motifs, and/or by comparing the structures or predicted structures of the proteins, e.g., by homology modeling.

Functional variants of the recombinant C11 hydroxylases, cytochrome P450 reductases, EPHs, SQEs, CDSs, and UGTs disclosed in this application are also encompassed by the present disclosure. For example, functional variants may bind one or more of the same substrates (e.g., mogrol, mogroside, or precursors thereof) or produce one or more of the same products (e.g., mogrol, mogroside, or precursors thereof). Functional variants may be identified using any method known in the art. For example, the algorithm of Karlin and Altschul Proc. Natl. Acad. Sci. USA 87:2264-68, 1990 described above may be used to identify homologous proteins with known functions.

Putative functional variants may also be identified by searching for polypeptides with functionally annotated domains. Databases including Pfam (Sonnhammer et al., Proteins. 1997 July; 28(3):405-20) may be used to identify polypeptides with a particular domain. For example, among oxidosqualene cyclases, additional CDS enzymes may be identified in some instances by searching for polypeptides with a leucine residue corresponding to position 123 of SEQ ID NO: 74. This leucine residue has been implicated in determining the product specificity of the CDS enzyme; mutation of this residue can, for instance, result in cycloartenol or parkeol as a product (Takase et al., Org Biomol Chem. 2015 Jul. 13(26):7331-6).

Additional UGT enzymes may be identified, for example, by searching for polypeptides with a UDPGT domain (PROSITE accession number PS00375).

Homology modeling may also be used to identify amino acid residues that are amenable to mutation without affecting function. A non-limiting example of such a method may include use of position-specific scoring matrix (PSSM) and an energy minimization protocol.

Position-specific scoring matrix (PSSM) uses a position weight matrix to identify consensus sequences (e.g., motifs). PSSM can be conducted on nucleic acid or amino acid sequences. Sequences are aligned, and the method takes into account the observed frequency of a particular residue (e.g., an amino acid or a nucleotide) at a particular position and the number of sequences analyzed. See, e.g., Stormo et al., Nucleic Acids Res. 1982 May 11; 10(9):2997-3011. The likelihood of observing a particular residue at a given position can be calculated. Without being bound by a particular theory, positions in sequences with high variability may be amenable to mutation (e.g., PSSM score ≥0) to produce functional homologs.

PSSM may be paired with calculation of a Rosetta energy function, which determines the difference between the wild-type and the single-point mutant. The Rosetta energy function calculates this difference as (ΔΔG_{calc}). With the Rosetta function, the bonding interactions between a mutated residue and the surrounding atoms are used to determine whether a mutation increases or decreases protein stability. For example, a mutation that is designated as favorable by the PSSM score (e.g. PSSM score ≥0), can then be analyzed using the Rosetta energy function to determine the potential impact of the mutation on protein stability. Without being bound by a particular theory, potentially stabilizing mutations are desirable for protein engineering (e.g., production of functional homologs). In some embodiments, a potentially stabilizing mutation has a ΔΔG_{calc} value of less than −0.1 (e.g., less than −0.2, less than −0.3, less than −0.35, less than −0.4, less than −0.45, less than −0.5, less than −0.55, less than −0.6, less than −0.65, less than −0.7, less than −0.75, less than −0.8, less than −0.85, less than −0.9, less than −0.95, or less than −1.0) Rosetta energy units (R.e.uz.). See, e.g., Goldenzweig et al., Mol Cell. 2016 Jul. 21; 63(2):337-346. doi: 10.1016/j.molcel.2016.06.012.

In some embodiments, a C11 hydroxylase, cytochrome P450 reductase, EPH, SQE, CDS, or UGT coding sequence comprises a mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 positions corresponding to a reference coding sequence. In some embodiments, the C11 hydroxylase, cytochrome P450 reductase, EPH, SQE, CDS, or UGT coding sequence comprises a mutation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more codons of the coding sequence relative to a reference coding sequence. As will be understood by one of ordinary skill in the art, a mutation within a codon may or may not change the amino acid that is encoded by the codon due to degeneracy of the genetic code. In some embodiments, the one or more mutations in the coding sequence do not alter the amino acid sequence of the coding sequence relative to the amino acid sequence of a reference polypeptide.

In some embodiments, the one or more mutations in a recombinant C11 hydroxylase, cytochrome P450 reductase, EPH, SQE, CDS, or UGT sequence alter the amino acid sequence of the polypeptide relative to the amino acid sequence of a reference polypeptide. In some embodiments, the one or more mutations alter the amino acid sequence of the recombinant polypeptide relative to the amino acid sequence of a reference polypeptide and alter (enhance or reduce) an activity of the polypeptide relative to the reference polypeptide.

The activity, including specific activity, of any of the recombinant polypeptides described in this application may be measured using routine methods. As a non-limiting example, a recombinant polypeptide's activity may be determined by measuring its substrate specificity, product(s) produced, the concentration of product(s) produced, or any combination thereof. As used in this application, “specific activity” of a recombinant polypeptide refers to the amount (e.g., concentration) of a particular product produced for a given amount (e.g., concentration) of the recombinant polypeptide per unit time.

The skilled artisan will also realize that mutations in a recombinant polypeptide coding sequence may result in conservative amino acid substitutions to provide functionally equivalent variants of the foregoing polypeptides, e.g., variants that retain the activities of the polypeptides. As used in this application, a “conservative amino acid substitution” refers to an amino acid substitution that does not alter the relative charge or size characteristics or functional activity of the protein in which the amino acid substitution is made.

In some instances, an amino acid is characterized by its R group (see, e.g., Table 2). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group includes lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

Non-limiting examples of functionally equivalent variants of polypeptides may include conservative amino acid substitutions in the amino acid sequences of proteins disclosed in this application. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. Additional non-limiting examples of conservative amino acid substitutions are provided in Table 2.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions.

TABLE 2
Non-limiting examples of conservative
amino acid substitutions

| Original | R Group | Conservative Amino |
|---|---|---|
| Residue | Type | Acid Substitutions |
| Ala | nonpolar aliphatic R group | Cys, Gly, Ser |
| Arg | positively charged R group | His, Lys |
| Asn | polar uncharged R group | Asp, Gln, Glu |
| Asp | negatively charged R group | Asn, Gln, Glu |
| Cys | polar uncharged R group | Ala, Ser |
| Gln | polar uncharged R group | Asn, Asp, Glu |
| Glu | negatively charged R group | Asn, Asp, Gln |
| Gly | nonpolar aliphatic R group | Ala, Ser |
| His | positively charged R group | Arg, Tyr, Trp |
| Ile | nonpolar aliphatic R group | Leu, Met, Val |
| Leu | nonpolar aliphatic R group | Ile, Met, Val |
| Lys | positively charged R group | Arg, His |
| Met | nonpolar aliphatic R group | Ile, Leu, Phe, Val |
| Pro | polar uncharged R group | |
| Phe | nonpolar aromatic R group | Met, Trp, Tyr |
| Ser | polar uncharged R group | Ala, Gly, Thr |
| Thr | polar uncharged R group | Ala, Asn, Ser |
| Trp | nonpolar aromatic R group | His, Phe, Tyr, Met |
| Tyr | nonpolar aromatic R group | His, Phe, Trp |
| Val | nonpolar aliphatic R group | Ile, Leu, Met, Thr |

Amino acid substitutions in the amino acid sequence of a polypeptide to produce a recombinant polypeptide variant having a desired property and/or activity can be made by alteration of the coding sequence of the polypeptide. Similarly, conservative amino acid substitutions in the amino acid sequence of a polypeptide to produce functionally equivalent variants of the polypeptide typically are made by alteration of the coding sequence of the recombinant polypeptide.

### Expression of Nucleic Acids in Host Cells

Aspects of the present disclosure relate to the recombinant expression of genes encoding enzymes, functional modifications and variants thereof, as well as uses relating thereto. For example, the methods described in this application may be used to produce mogrol precursors, mogrol and/or mogrosides.

The term “heterologous” with respect to a polynucleotide, such as a polynucleotide comprising a gene, is used interchangeably with the term “exogenous” and the term “recombinant” and refers to: a polynucleotide that has been artificially supplied to a biological system; a polynucleotide that has been modified within a biological system, or a polynucleotide whose expression or regulation has been manipulated within a biological system. A heterologous polynucleotide that is introduced into or expressed in a host cell may be a polynucleotide that comes from a different organism or species from the host cell, or may be a synthetic polynucleotide, or may be a polynucleotide that is also endogenously expressed in the same organism or species as the host cell. For example, a polynucleotide that is endogenously expressed in a host cell may be considered heterologous when it is situated non-naturally in the host cell; expressed recombinantly in the host cell, either stably or transiently; modified within the host cell; selectively edited within the host cell; expressed in a copy number that differs from the naturally occurring copy number within the host cell; or expressed in a non-natural way within the host cell, such as by manipulating regulatory regions that control expression of the polynucleotide. In some embodiments, a heterologous polynucleotide is a polynucleotide that is endogenously expressed in a host cell but whose expression is driven by a promoter that does not naturally regulate expression of the polynucleotide. In other embodiments, a heterologous polynucleotide is a polynucleotide that is endogenously expressed in a host cell and whose expression is driven by a promoter that does naturally regulate expression of the polynucleotide, but the promoter or another regulatory region is modified. In some embodiments, the promoter is recombinantly activated or repressed. For example, gene-editing based techniques may be used to regulate expression of a polynucleotide, including an endogenous polynucleotide, from a promoter, including an endogenous promoter. See, e.g., Chavez et al., Nat Methods. 2016 July; 13(7): 563-567. A heterologous polynucleotide may comprise a wild-type sequence or a mutant sequence as compared with a reference polynucleotide sequence.

A nucleic acid encoding any of the recombinant polypeptides, such as C11 hydroxylases, cytochrome P450 reductases, EPHs, SQEs, CDSs, or UGTs described in this application may be incorporated into any appropriate vector through any method known in the art. For example, the vector may be an expression vector, including but not limited to a viral vector (e.g., a lentiviral, retroviral, adenoviral, or adeno-associated viral vector), any vector suitable for transient expression, any vector suitable for constitutive expression, or any vector suitable for inducible expression (e.g., a galactose-inducible or doxycycline-inducible vector).

In some embodiments, a vector replicates autonomously in the cell. A vector can contain one or more endonuclease restriction sites that are cut by a restriction endonuclease to insert and ligate a nucleic acid containing a gene described in this application to produce a recombinant vector that is able to replicate in a cell. Vectors are typically composed of DNA, although RNA vectors are also available. Cloning vectors include, but are not limited to, plasmids, fosmids, phagemids, virus genomes and artificial chromosomes. As used in this application, the terms “expression vector” or “expression construct” refer to a nucleic acid construct, generated recombinantly or synthetically, with a series of specified nucleic acid elements that permit transcription of a particular nucleic acid in a host cell such as a yeast cell. In some embodiments, the nucleic acid sequence of a gene described in this application is inserted into a cloning vector such that it is operably joined to regulatory sequences and, in some embodiments, expressed as an RNA transcript. In some embodiments, the vector contains one or more markers, such as a selectable marker as described in this application, to identify cells transformed or transfected with the recombinant vector.

In some embodiments, the nucleic acid sequence of a gene described in this application is re-coded. Re-coding may increase production of the gene product by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, including all values in between) relative to a reference sequence that is not re-coded.

A coding sequence and a regulatory sequence are said to be “operably joined” when the coding sequence and the regulatory sequence are covalently linked and the expression or transcription of the coding sequence is under the influence or control of the regulatory sequence. If the coding sequence is to be translated into a functional protein, the coding sequence and the regulatory sequence are said to be operably joined if induction of a promoter in the 5′ regulatory sequence permits the coding sequence to be transcribed and if the nature of the linkage between the coding sequence and the regulatory sequence does not (1) result in the introduction of a frame-shift mutation, (2) interfere with the ability of the promoter region to direct the transcription of the coding sequence, or (3) interfere with the ability of the corresponding RNA transcript to be translated into a protein.

In some embodiments, the nucleic acid encoding any of the proteins described in this application is under the control of regulatory sequences (e.g., enhancer sequences). In some embodiments, a nucleic acid is expressed under the control of a promoter. The promoter can be a native promoter, e.g., the promoter of the gene in its endogenous context, which provides normal regulation of expression of the gene. Alternatively, a promoter can be a promoter that is different from the native promoter of the gene, e.g., the promoter is different from the promoter of the gene in its endogenous context.

In some embodiments, the promoter is a eukaryotic promoter. Non-limiting examples of eukaryotic promoters include TDH3, PGK1, PKC1, PDC1, TEF1, TEF2, RPL18B, SSA1, TDH2, PYK1, TPI1 GAL1, GAL10, GAL7, GAL3, GAL2, MET3, MET25, HXT3, HXT7, ACT1, ADH1, ADH2, CUP1-1, ENO2, pAOX1, pGAP1, and SOD1, as would be known to one of ordinary skill in the art (see, e.g., Addgene website: blog.addgene.org/plasmids-101-the-promoter-region). In some embodiments, the promoter is a prokaryotic promoter (e.g., bacteriophage or bacterial promoter). Non-limiting examples of bacteriophage promoters include Pls1con, T3, T7, SP6, and PL. Non-limiting examples of bacterial promoters include Pbad, PmgrB, Ptrc2, Plac/ara, Ptac, and Pm.

In some embodiments, the promoter is an inducible promoter. As used in this application, an “inducible promoter” is a promoter controlled by the presence or absence of a molecule. Non-limiting examples of inducible promoters include chemically regulated promoters and physically regulated promoters. For chemically regulated promoters, the transcriptional activity can be regulated by one or more compounds, such as alcohol, tetracycline, galactose, a steroid, a metal, or other compounds. For physically regulated promoters, transcriptional activity can be regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein (tTA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination thereof.

In some embodiments, the promoter is a constitutive promoter. As used in this application, a “constitutive promoter” refers to an unregulated promoter that allows continuous transcription of a gene. Non-limiting examples of a constitutive promoter include TDH3, PGK1, PKC1, PDC1, TEF1, TEF2, RPL18B, SSA1, TDH2, PYK1, TPI1, HXT3, HXT7, ACT1, ADH1, ADH2, ENO2, pGAP1, and SOD1.

Other inducible promoters or constitutive promoters known to one of ordinary skill in the art are also contemplated.

The precise nature of the regulatory sequences needed for gene expression may vary between species or cell types, but generally include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences. The vectors disclosed in this application may include 5′ leader or signal sequences. The regulatory sequence may also include a terminator sequence. In some embodiments, a terminator sequence marks the end of a gene in DNA during transcription. The choice and design of one or more appropriate vectors suitable for inducing expression of one or more genes described in this application in a host cell is within the ability and discretion of one of ordinary skill in the art.

Expression vectors containing the necessary elements for expression are commercially available and known to one of ordinary skill in the art (see, e.g., Sambrook et al., Molecular Cloning: A Laboratory Manual, Fourth Edition, Cold Spring Harbor Laboratory Press, 2012).

In some embodiments, introduction of a nucleic acid encoding any of the recombinant polypeptides results in genomic integration of the nucleic acid. In some embodiments, a host cell comprises at least 1 copy, at least 2 copies, at least 3 copies, at least 4 copies, at least 5 copies, at least 6 copies, at least 7 copies, at least 8 copies, at least 9 copies, at least 10 copies, at least 11 copies, at least 12 copies, at least 13 copies, at least 14 copies, at least 15 copies, at least 16 copies, at least 17 copies, at least 18 copies, at least 19 copies, at least 20 copies, at least 21 copies, at least 22 copies, at least 23 copies, at least 24 copies, at least 25 copies, at least 26 copies, at least 27 copies, at least 28 copies, at least 29 copies, at least 30 copies, at least 31 copies, at least 32 copies, at least 33 copies, at least 34 copies, at least 35 copies, at least 36 copies, at least 37 copies, at least 38 copies, at least 39 copies, at least 40 copies, at least 41 copies, at least 42 copies, at least 43 copies, at least 44 copies, at least 45 copies, at least 46 copies, at least 47 copies, at least 48 copies, at least 49 copies, at least 50 copies, at least 60 copies, at least 70 copies, at least 80 copies, at least 90 copies, at least 100 copies, or more, including any values in between, of a nucleotide sequence encoding any of the recombinant polypeptides in its genome.

In some instances, a host cell comprises at least two different C11 hydroxylases, cytochrome P450 reductases, EPHs, SQEs, CDSs, or UGTs.

In some instances, a host cell comprises: an upregulated squalene synthase, an upregulated SQE, a downregulated lanosterol synthase, at least one C11 hydroxylase, at least one cytochrome P450 reductase, at least one CDS, and at least one EPH. In some instances, a host cell comprises: an upregulated SQE, at least one CDS, at least one epoxide hydrolase, at least one C11 hydroxylase, and/or at least one cytochrome P450 reductase. In some instances, a host cell comprises: an upregulated SQE, at least one CDS, at least one C11 hydroxylase, and at least one cytochrome P450 reductase. In some instances, a host cell further comprises at least one epoxide hydrolase. In some instances, a host cell comprises two different C11 hydroxylases and two different cytochrome P450 reductases. In some instances, a squalene synthase is ERG9. In some instances, a squalene epoxidase is ERG1. In some instances, a lanosterol synthase is ERG7. In some instances, a C11 hydroxylase is CYP1798. In some instances, a C11 hydroxylase is any of the C11 hydroxylases described in this application. In some instances, a C11 hydroxylase is PGA2Id23-129_CYP5491-T351M (SEQ ID NO: 305). In some instances, an epoxide hydrolase is EPH3. In some instances, an epoxide hydrolase is EPH2. In some instances, a cytochrome P450 reductase is AtCPR1. In some instances, a cytochrome P450 reductase is CPR4497. In some embodiments, a host cell comprises AtCPR1 and CPR4497. In some instances, a cytochrome P450 reductase is CPR4497. In some instances, a CDS is Siraitia CDS (sgCDS) or SEQ ID NO: 66.

In some instances, a host cell comprises: an upregulated ERG9, an upregulated ERG1; a downregulated ERG7; at least one (e.g., 1, 2, 3, 4, 5, 6, 7, 8, 9, or 10) copy of a nucleotide sequence encoding CYP1798; at least one (e.g., 1, 2, 3, 4, 5, 6, 7, 8, 9, or 10) copy of a nucleotide sequence encoding AtCPR1; at least one (e.g., 1, 2, 3, 4, 5, 6, 7, 8, 9, or 10) copy of a nucleotide sequence encoding CPR4497; at least one (e.g., 1, 2, 3, 4, 5, 6, 7, 8, 9, or 10) copy of a nucleotide sequence encoding sgCDS; at least one (e.g., 1, 2, 3, 4, 5, 6, 7, 8, 9, or 10) copy of a nucleotide sequence encoding EPH3; and/or at least one (e.g., 1, 2, 3, 4, 5, 6, 7, 8, 9, or 10) copy of a nucleotide sequence encoding atEPH2. See, e.g., Table 6, which provides non-limiting examples of CYP1798, AtCPR1, CPR4497, sgCDS, EPH3, atEPH2, ERG9, ERG1, and ERG7.

As used in this application, an “upregulated” enzyme is an enzyme whose expression is increased relative to a control. Expression of an enzyme can be upregulated using any means known to one of ordinary skill in the art. In some embodiments, expression of an enzyme is upregulated by selecting a specific promoter to control expression of the enzyme and/or by engineering the promoter of the enzyme. In some embodiments, expression of an enzyme is upregulated by expression of multiple copies of the enzyme in a host cell. In some embodiments, expression of an enzyme in a host cell is upregulated relative to a control host cell. In some embodiments, a control host cell is a host cell that does not comprise a heterologous nucleic acid encoding the enzyme. In some embodiments, a control host cell is a host cell that comprises one copy of a heterologous nucleic acid encoding the enzyme. In some embodiments, a control host cell is a host cell that comprises less copies of a heterologous nucleic acid encoding the enzyme than a control host cell in which the enzyme is upregulated. In some embodiments, a control host cell is a host cell in which expression of the enzyme is controlled by a different promoter than in the host cell in which the enzyme is upregulated. In some embodiments, expression of an enzyme is upregulated by at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1,000% relative to a control.

### Host Cells

Any of the proteins or enzymes of the disclosure may be expressed in a host cell. The term “host cell” refers to a cell that can be used to express a polynucleotide, such as a polynucleotide that encodes an enzyme used in production of mogrol, mogrosides, and precursors thereof.

Any suitable host cell may be used to produce any of the recombinant polypeptides, including C11 hydroxylases, cytochrome P450 reductases, EPH, SQEs, CDSs, and UGTs disclosed in this application, including eukaryotic cells or prokaryotic cells. Suitable host cells include, but are not limited to, fungal cells (e.g., yeast cells), bacterial cells (e.g., E. coli cells), algal cells, plant cells, insect cells, and animal cells, including mammalian cells. Suitable yeast host cells include, but are not limited to, Candida, Escherichia, Hansenula, Saccharomyces (e.g., S. cerevisiae), Schizosaccharomyces, Pichia, Kluyveromyces (e.g., K. lactis), and Yarrowia. In some embodiments, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, or Yarrowia lipolytica.

In some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In certain embodiments, the host cell is an algal cell such as, Chlamydomonas (e.g., C. Reinhardtii) and Phormidium (P. sp. ATCC29409).

In other embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas.

In some embodiments, the bacterial host cell is of the Agrobacterium species (e.g., A. radiobacter, A. rhizogenes, A. rubi), the Arthrobacter species (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), or the Bacillus species (e.g., B. thuringiensis, B. anthracis, B. megaterium, B. subtilis, B. lentus, B. circulans, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell is an industrial Bacillus strain including but not limited to B. subtilis, B. pumilus, B. licheniformis, B. megaterium, B. clausii, B. stearothermophilus and B. amyloliquefaciens. In some embodiments, the host cell is an industrial Clostridium species (e.g., C. acetobutylicum, C. tetani E88, C. lituseburense, C. saccharobutylicum, C. perfringens, C. beijerinckii). In some embodiments, the host cell is an industrial Corynebacterium species (e.g., C. glutamicum, C. acetoacidophilum). In some embodiments, the host cell is an industrial Escherichia species (e.g., E. coli). In some embodiments, the host cell is an industrial Erwinia species (e.g., E. uredovora, E. carotovora, E. ananas, E. herbicola, E. punctata, E. terreus). In some embodiments, the host cell is an industrial Pantoea species (e.g., P. citrea, P. agglomerans). In some embodiments, the host cell is an industrial Pseudomonas species, (e.g., P. putida, P. aeruginosa, P. mevalonii). In some embodiments, the host cell is an industrial Streptococcus species (e.g., S. equisimiles, S. pyogenes, S. uberis). In some embodiments, the host cell is an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell is an industrial Zymomonas species (e.g., Z. mobilis, Z. lipolytica).

The present disclosure is also suitable for use with a variety of animal cell types, including mammalian cells, for example, human (including 293, HeLa, WI38, PER.C6 and Bowes melanoma cells), mouse (including 3T3, NS0, NS1, Sp2/0), hamster (CHO, BHK), monkey (COS, FRhL, Vero), and hybridoma cell lines.

The present disclosure is also suitable for use with a variety of plant cell types.

The term “cell,” as used in this application, may refer to a single cell or a population of cells, such as a population of cells belonging to the same cell line or strain. Use of the singular term “cell” should not be construed to refer explicitly to a single cell rather than a population of cells.

The host cell may comprise genetic modifications relative to a wild-type counterpart. As a non-limiting example, a host cell (e.g., S. cerevisiae) may be modified to reduce or inactivate one or more of the following genes: hydroxymethylglutaryl-CoA (HMG-CoA) reductase (HMG1), acetyl-CoA C-acetyltransferase (acetoacetyl-CoA thiolase) (ERG10), 3-hydroxy-3-methylglutaryl-CoA (HMG-CoA) synthase (ERG13), farnesyl-diphosphate farnesyl transferase (squalene synthase) (ERG5), may be modified to overexpress squalene epoxidase (ERG1), or may be modified to downregulate lanosterol synthase (ERG7).

Reduction of gene expression and/or gene inactivation may be achieved through any suitable method, including but not limited to deletion of the gene, introduction of a point mutation into the endogenous gene, and/or truncation of the endogenous gene. For example, polymerase chain reaction (PCR)-based methods may be used (see, e.g., Gardner et al., Methods Mol Biol. 2014; 1205:45-78) or well-known gene-editing techniques may be used. As a non-limiting example, genes may be deleted through gene replacement (e.g., with a marker, including a selection marker). A gene may also be truncated through the use of a transposon system (see, e.g., Poussu et al., Nucleic Acids Res. 2005; 33(12): e104).

A vector encoding any of the recombinant polypeptides described in this application may be introduced into a suitable host cell using any method known in the art. Non-limiting examples of yeast transformation protocols are described in Gietz et al., Yeast transformation can be conducted by the LiAc/SS Carrier DNA/PEG method. Methods Mol Biol. 2006; 313:107-20, which is incorporated by reference in its entirety. Host cells may be cultured under any conditions suitable as would be understood by one of ordinary skill in the art. For example, any media, temperature, and incubation conditions known in the art may be used. For host cells carrying an inducible vector, cells may be cultured with an appropriate inducible agent to promote expression.

Any of the cells disclosed in this application can be cultured in media of any type (rich or minimal) and any composition prior to, during, and/or after contact and/or integration of a nucleic acid. The conditions of the culture or culturing process can be optimized through routine experimentation as would be understood by one of ordinary skill in the art. In some embodiments, the selected media is supplemented with various components. In some embodiments, the concentration and amount of a supplemental component is optimized. In some embodiments, other aspects of the media and growth conditions (e.g., pH, temperature, etc.) are optimized through routine experimentation. In some embodiments, the frequency that the media is supplemented with one or more supplemental components, and the amount of time that the cell is cultured, is optimized.

Culturing of the cells described in this application can be performed in culture vessels known and used in the art. In some embodiments, an aerated reaction vessel (e.g., a stirred tank reactor) is used to culture the cells. In some embodiments, a bioreactor or fermentor is used to culture the cell. Thus, in some embodiments, the cells are used in fermentation. As used in this application, the terms “bioreactor” and “fermentor” are interchangeably used and refer to an enclosure, or partial enclosure, in which a biological, biochemical and/or chemical reaction takes place, involving a living organism, part of a living organism, or purified enzymes. A “large-scale bioreactor” or “industrial-scale bioreactor” is a bioreactor that is used to generate a product on a commercial or quasi-commercial scale. Large scale bioreactors typically have volumes in the range of liters, hundreds of liters, thousands of liters, or more.

Non-limiting examples of bioreactors include: stirred tank fermentors, bioreactors agitated by rotating mixing devices, chemostats, bioreactors agitated by shaking devices, airlift fermentors, packed-bed reactors, fixed-bed reactors, fluidized bed bioreactors, bioreactors employing wave induced agitation, centrifugal bioreactors, roller bottles, and hollow fiber bioreactors, roller apparatuses (for example benchtop, cart-mounted, and/or automated varieties), vertically-stacked plates, spinner flasks, stirring or rocking flasks, shaken multi-well plates, MD bottles, T-flasks, Roux bottles, multiple-surface tissue culture propagators, modified fermentors, and coated beads (e.g., beads coated with serum proteins, nitrocellulose, or carboxymethyl cellulose to prevent cell attachment).

In some embodiments, the bioreactor includes a cell culture system where the cell (e.g., yeast cell) is in contact with moving liquids and/or gas bubbles. In some embodiments, the cell or cell culture is grown in suspension. In other embodiments, the cell or cell culture is attached to a solid phase carrier. Non-limiting examples of a carrier system includes microcarriers (e.g., polymer spheres, microbeads, and microdisks that can be porous or non-porous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can comprising porous fibers), microcarriers having reduced ion exchange capacity, encapsulation cells, capillaries, and aggregates. In some embodiments, carriers are fabricated from materials such as dextran, gelatin, glass, or cellulose.

In some embodiments, industrial-scale processes are operated in continuous, semi-continuous or non-continuous modes. Non-limiting examples of operation modes are batch, fed batch, extended batch, repetitive batch, draw/fill, rotating-wall, spinning flask, and/or perfusion mode of operation. In some embodiments, a bioreactor allows continuous or semi-continuous replenishment of the substrate stock, for example a carbohydrate source and/or continuous or semi-continuous separation of the product, from the bioreactor.

In some embodiments, the bioreactor or fermentor includes a sensor and/or a control system to measure and/or adjust reaction parameters. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, such as oxygen concentration and CO₂ concentration, nutrient concentrations, metabolite concentrations, concentration of an oligopeptide, concentration of an amino acid, concentration of a vitamin, concentration of a hormone, concentration of an additive, serum concentration, ionic strength, concentration of an ion, relative humidity, molarity, osmolarity, concentration of other chemicals, for example buffering agents, adjuvants, or reaction by-products), physical/mechanical parameters (e.g., density, conductivity, degree of agitation, pressure, and flow rate, shear stress, shear rate, viscosity, color, turbidity, light absorption, mixing rate, conversion rate, as well as thermodynamic parameters, such as temperature, light intensity/quality, etc.). Sensors to measure the parameters described in this application are well known to one of ordinary skill in the relevant mechanical and electronic arts. Control systems to adjust the parameters in a bioreactor based on the inputs from a sensor described in this application are well known to one of ordinary skill in the art in bioreactor engineering.

In some embodiments, the method involves batch fermentation (e.g., shake flask fermentation). General considerations for batch fermentation (e.g., shake flask fermentation) include the level of oxygen and glucose. For example, batch fermentation (e.g., shake flask fermentation) may be oxygen and glucose limited, so in some embodiments, the capability of a strain to perform in a well-designed fed-batch fermentation is underestimated. Also, the final product (e.g., mogrol precursor, mogrol, mogroside precursor, or mogroside) may display some differences from the substrate (e.g., mogrol precursor, mogrol, mogroside precursor, or mogroside) in terms of solubility, toxicity, cellular accumulation and secretion and in some embodiments can have different fermentation kinetics.

The methods described in this application encompass production of mogrol precursors (e.g., squalene, 2,3-oxidosqualene, or 24-25 epoxy-cucurbitadienol), mogrol, or mogrosides (e.g., MIA1, MIE1, MIIA1, MIIA2, MIIIA1, MIIE, MIII, siamenoside I, mogroside IV, isomogroside IV, MIIIE, and mogroside V) using a recombinant cell, cell lysate or isolated recombinant polypeptides, including C11 hydroxylases, cytochrome P450 reductases, EPHs, SQEs, CDSs, and UGTs.

Mogrol precursors (e.g., squalene, 2,3-oxidosqualene, or 24-25 epoxy-cucurbitadienol), mogrol, mogrosides (e.g., MIA1, MIE, MIIA1, MIIA2, MIIIA1, MIIE, MIII, siamenoside I, mogroside IV, isomogroside IV, MIIIE, and mogroside V) produced by any of the recombinant cells disclosed in this application may be identified and extracted using any method known in the art. Mass spectrometry (e.g., LC-MS, GC-MS) is a non-limiting example of a method for identification and may be used to help extract of a compound of interest.

The phraseology and terminology used in this a is for the purpose of description and should not be regarded as limiting. The use of terms such as “including,” “comprising,” “having,” “containing,” “involving,” and/or variations thereof in this application, is meant to encompass the items listed thereafter and equivalents thereof as well as additional items.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Example 1: Identification of C11 Hydroxylases for Mogrol Production

### Development of a C11 Hydroxylase Screening Library

Screening was conducted to identify C11 hydroxylases that may be useful in the production of mogrol. A library containing 1,190 proteins was screened in vivo for the target product mogrol. The library comprised variants of CYP5491 including single substitution mutations and signal recognition particle (SRP)-dependent signal peptide and transmembrane domain (TM)-CYP5491 fusions (e.g., as depicted in FIG. 2B). The library also included close homologs of CYP5491.

For the single substitution mutations targeting the active site of CYP5491, 37 residues were identified around a modeled lanosterol (FIGS. 4A-4B). Lanosterol is chemically similar to mogrol and was modeled in the active site of the CYP5491 homology model. Residues hypothesized to interact or facilitate interactions with lanosterol were chosen for scanning mutagenesis. Saturation mutagenesis of these residues was conducted. Protein stabilization mutations included single amino acid substitutions that were indicated to have a stabilizing effect by Rosetta. To identify positions to mutate for the creation of Rosetta stabilization mutations, positions were selected based on conservation in a multiple sequence alignment. Rosetta energy calculation was used to screen observed mutations in silico.

The C11 hydroxylase library plasmid structure is depicted in FIG. 2C. The promoter (˜700 bp) and terminator (˜250 bp) were used as the homology arm for genome integration.

S. cerevisiae host cells were used for the screens. The host cell base strain was engineered to express one or more copies of CYP1798, AtCPR1, CPR4497, sgCDS, EPH3, and atEPH2, as well as to upregulate expression of ERG5 and ERG1 and downregulate expression of ERG7. The base strain also had several copies of pPGK1_X_tSSA1 integrated into the genome. “X” corresponds to the F-Cph1 recognition site, which is 24 bp and has the sequence GATGCACGAGCGCAACGCTCACAA (SEQ ID NO: 415).

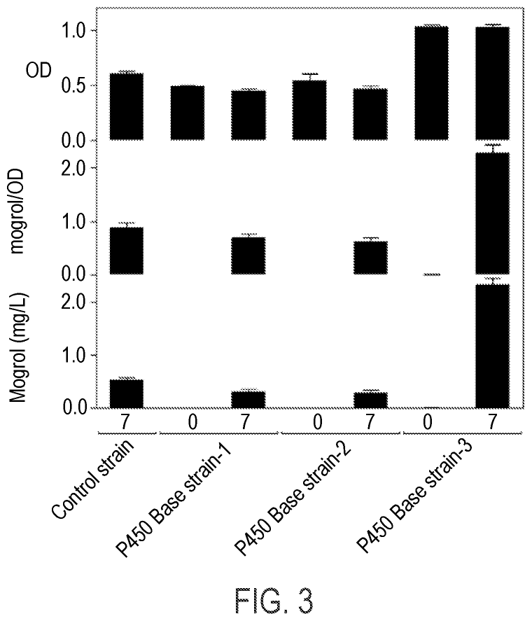

CYP5491 was used as a positive control for P450 screening. The control strain, shown in FIG. 3, contains multiple copies of CYP5491. The control strain also comprises a CDS, an EPH, two cytochrome P450 reductases, and an upregulated SQE. Base strains with no copies of CYP5491 were used as negative controls. P450 base strain-1 was used for the library screening.

For each candidate C11 hydroxylase to be tested, multiple copies of a nucleotide sequence encoding the candidate C11 hydroxylase were integrated into the genome of the S. cerevisiae host cell. Cells were transformed with the construct of interest using LiAc-mediated transformation.

### Screening Results

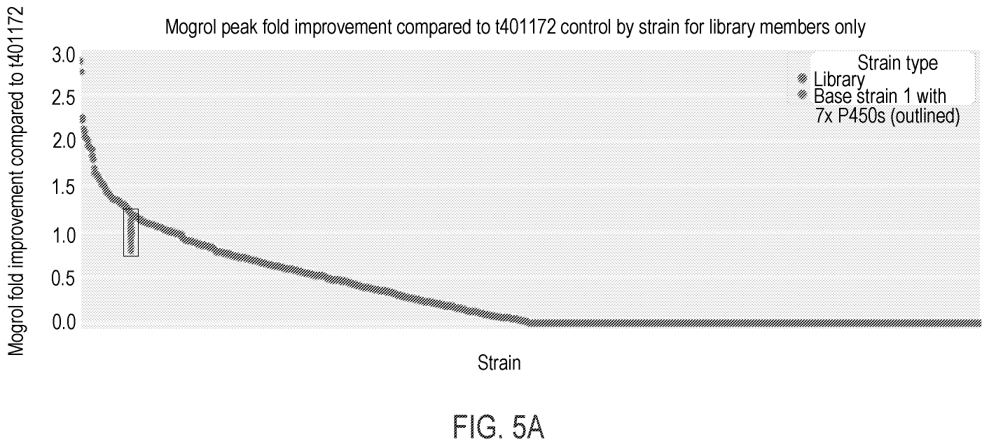

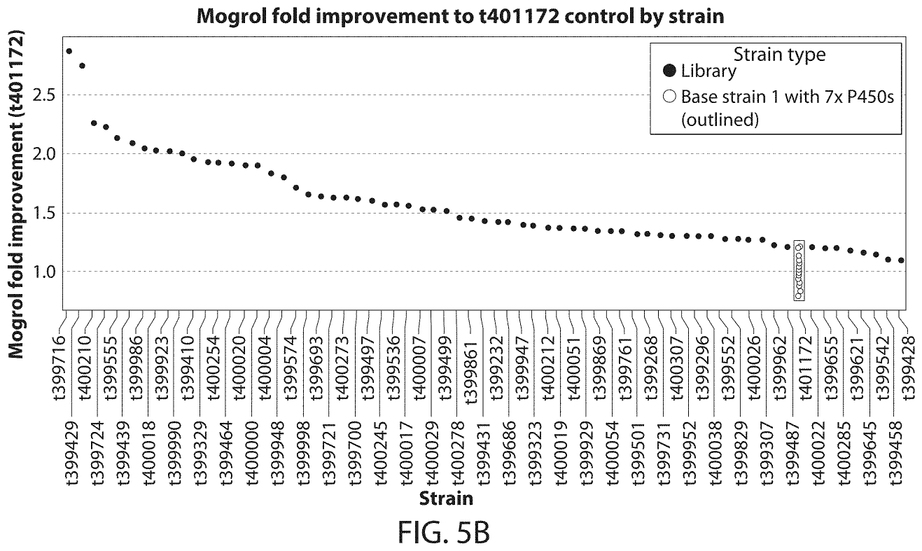

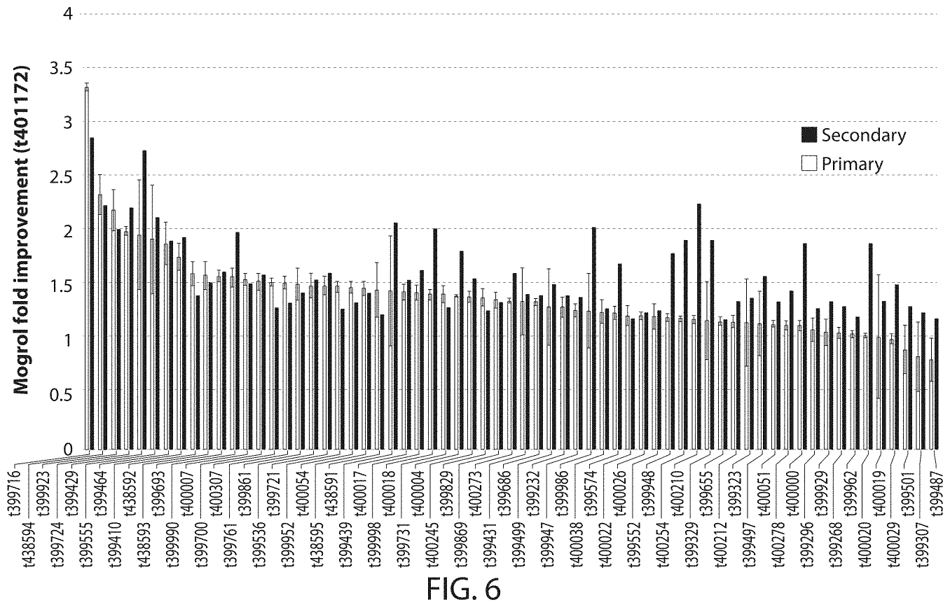

A candidate in the C11 hydroxylase screening library was considered a hit if the candidate resulted in production of mogrol that was 1.2 fold greater than the production of mogrol by the control base strain comprising wild-type CYP5491 (SEQ ID NO: 208). As shown in FIG. 5A, multiple candidates in the C11 hydroxylase screening library resulted in more than two times the mogrol production of wild-type CYP5491 (SEQ ID NO: 208). FIG. 5B shows 66 members of the library that were considered hits. 66 hits and 5 additional candidates that performed similarly to the control strain from this screen were subsequently run in another library screen with 4 replicates. The additional candidates that performed similarly to the control strain were included for assay validation. Hits from the initial screen were confirmed (see, e.g., Table 3 below).

FIG. 6 shows a comparison between the two screens. Top hits were consistent in both screens.

For the active site mutants, hits were identified at 20 unique positions, with six positions identified through at least two mutations. Mutation hotspots were identified near the active site entrance and around the heme group. In these experiments, a residue was designated a mutation hotspot if multiple different variants with substitutions at that residue showed an activity benefit. Measurement of the fold increase in mogrol production for CYP5491 active site mutants relative to wild-type CYP5491 (SEQ ID NO: 208) is shown in Table 3.

Table 4 shows the nucleic acid and amino acid sequences of signal peptides and CYP5491 fusions, as well as the fold increase in mogrol production for CYP5491 fusions relative to wild-type CYP5491 (SEQ ID NO: 208).

A CYP5491 fusion comprising a signal peptide from ERG11 was identified as a hit in the screen. The CYP5491 fusion comprised the first 25 amino acid residues from ERG11 and residues 3-473 from wild-type CYP5491. The amino acid sequence of this ERG11 N-terminal-CYP5491 fusion is provided as SEQ ID NO: 280.

TABLE 3
Mogrol production by CYP5491 mutants
relative to wild-type CYP5491

| | fold- | fold- |
|---|---|---|
| CYP5491 | increase- | increase- |
| Mutant | mean | std |
| F147L | 1.407273 | 0.049504 |
| A85S | 1.18231 | 0.055106 |
| T470E | 1.148014 | 0.044508 |
| D299A | 1.209386 | 0.022449 |
| K185H | 1.202166 | 0.039326 |
| H160E | 1.370036 | 0.018989 |
| A140P | 1.166065 | 0.027335 |
| T117G | 1.543321 | 0.038376 |
| S155A | 1.485019 | 0.063376 |
| S49A | 1.193681 | 0.35601 |
| S49F | 1.973901 | 0.499373 |
| S49H | 0.833791 | 0.202398 |
| S49I | 1.461538 | 0.50322 |
| S49M | 1.774725 | 0.128936 |
| L76I | 1.225275 | 0.114201 |
| L76V | 1.25 | 0.166264 |
| D107P | 1.104396 | 0.069356 |
| D107R | 1.108516 | 0.107705 |
| L109A | 1.512363 | 0.113439 |
| L109C | 0.861264 | 0.316093 |
| L109F | 1.313187 | 0.352249 |
| L109W | 1.934066 | 0.497103 |
| L109Y | 1.163462 | 0.294093 |
| F112T | 1.167582 | 0.141903 |
| F112W | 0.921703 | 0.219385 |
| L354V | 1.173077 | 0.399616 |
| M376A | 1.364011 | 0.30567 |
| M376C | 0.896978 | 0.493454 |
| I458P | 1.899725 | 0.193605 |
| V57A | 1.223636 | 0.066224 |
| W119R | 1.547273 | 0.077737 |
| L120H | 1.429091 | 0.075697 |
| L120N | 1.183636 | 0.041726 |
| L210S | 1.087273 | 0.120018 |
| S211N | 1.598182 | 0.052905 |
| A282V | 2.203636 | 0.189742 |
| V350F | 1.378182 | 0.073331 |
| V350I | 1.523636 | 0.151917 |
| V350L | 1.538182 | 0.053936 |
| V350M | 1.363636 | 0.034879 |
| T351L | 2.010909 | 0.042199 |
| T351M | 3.332727 | 0.037262 |
| A353G | 1.510909 | 0.085099 |
| L354I | 1.401818 | 0.07953 |
| L212F | 1.259928 | 0.058509 |
| S49L | 2.342697 | 0.181161 |

TABLE 4
Mogrol production by CYP5491 fusions relative to wild-type CYP5491

| Signal | | | Fusion | | Fold | Fold |
|---|---|---|---|---|---|---|
| Peptide | Signal Peptide | Signal Peptide | Sequence | Fusion | Improve- | Improve- |
| UniProt | AA | Nucleotide | (amino | Sequence | ment | ment |
| ID | Sequence | Sequence | acid) | (nt)* | (mean) | (st dev) |
| P40367 | SEQ ID NO: 209 | SEQ ID NO: 233 | SEQ ID | SEQ ID NO: 281 | 1.021818 | 0.044635 |
| | | | NO: 257 | | | |
| Q12438 | SEQ ID NO: 210 | SEQ ID NO: 234 | SEQ ID | SEQ ID NO: 282 | 1.452727 | 0.067803 |
| | | | NO: 258 | | | |
| P38286 | SEQ ID NO: 211 | SEQ ID NO: 235 | SEQ ID | SEQ ID NO: 283 | 1.285455 | 0.060083 |
| | | | NO: 259 | | | |
| Q04121 | SEQ ID NO: 212 | SEQ ID NO: 236 | SEQ ID | SEQ ID NO: 284 | 1.22 | 0.036303 |
| | | | NO: 260 | | | |
| P38992 | SEQ ID NO: 213 | SEQ ID NO: 237 | SEQ ID | SEQ ID NO: 285 | 1.041818 | 0.566569 |
| | | | NO: 261 | | | |
| P38954 | SEQ ID NO: 214 | SEQ ID NO: 238 | SEQ ID | SEQ ID NO: 286 | 1.158182 | 0.033262 |
| | | | NO: 262 | | | |
| Q12270 | SEQ ID NO: 215 | SEQ ID NO: 239 | SEQ ID | SEQ ID NO: 287 | 1.146209 | 0.045042 |
| | | | NO: 263 | | | |
| Pl6474 | SEQ ID NO: 216 | SEQ ID NO: 240 | SEQ ID | SEQ ID NO: 288 | 1.276173 | 0.342047 |
| | | | NO: 264 | | | |
| Q04969 | SEQ ID NO: 217 | SEQ ID NO: 241 | SEQ ID | SEQ ID NO: 289 | 1.418773 | 0.012506 |
| | | | NO: 265 | | | |
| P14359 | SEQ ID NO: 218 | SEQ ID NO: 242 | SEQ ID | SEQ ID NO: 290 | 1.052347 | 0.02156 |
| | | | NO: 266 | | | |
| P38841 | SEQ ID NO: 219 | SEQ ID NO: 243 | SEQ ID | SEQ ID NO: 291 | 1.068592 | 0.030633 |
| | | | NO: 267 | | | |
| Q07451 | SEQ ID NO: 220 | SEQ ID NO: 244 | SEQ ID | SEQ ID NO: 292 | 1.494585 | 0.050369 |
| | | | NO: 268 | | | |
| P36057 | SEQ ID NO: 221 | SEQ ID NO: 245 | SEQ ID | SEQ ID NO: 293 | 1.5 | 0.049455 |
| | | | NO: 269 | | | |
| P40071 | SEQ ID NO: 222 | SEQ ID NO: 246 | SEQ ID | SEQ ID NO: 294 | 1.236462 | 0.090445 |
| | | | NO: 270 | | | |
| P16603 | SEQ ID NO: 223 | SEQ ID NO: 247 | SEQ ID | SEQ ID NO: 295 | 1.431408 | 0.048029 |
| | | | NO: 271 | | | |
| P39704 | SEQ ID NO: 224 | SEQ ID NO: 248 | SEQ ID | SEQ ID NO: 296 | 1.588448 | 0.088035 |
| | | | NO: 272 | | | |
| Q06689 | SEQ ID NO: 225 | SEQ ID NO: 249 | SEQ ID | SEQ ID NO: 297 | 1.563177 | 0.051563 |
| | | | NO: 273 | | | |
| P53903 | SEQ ID NO: 226 | SEQ ID NO: 250 | SEQ ID | SEQ ID NO: 298 | 1.447653 | 0.068115 |
| | | | NO: 274 | | | |
| P53154 | SEQ ID NO: 227 | SEQ ID NO: 251 | SEQ ID | SEQ ID NO: 299 | 1.268953 | 0.109876 |
| | | | NO: 275 | | | |
| P38626 | SEQ ID NO: 228 | SEQ ID NO: 252 | SEQ ID | SEQ ID NO: 300 | 1.234657 | 0.033866 |
| | | | NO: 276 | | | |
| P38353 | SEQ ID NO: 229 | SEQ ID NO: 253 | SEQ ID | SEQ ID NO: 301 | 1.602996 | 0.130317 |
| | | | NO: 277 | | | |
| P38310 | SEQ ID NO: 230 | SEQ ID NO: 254 | SEQ ID | SEQ ID NO: 302 | 1.617978 | 0.112276 |
| | | | NO: 278 | | | |
| P53045 | SEQ ID NO: 231 | SEQ ID NO: 255 | SEQ ID | SEQ ID NO: 303 | 1.473783 | 0.247408 |
| | | | NO: 279 | | | |
| P10614 | SEQ ID NO: 232 | SEQ ID NO: 256 | SEQ ID | SEQ ID NO: 304 | 1.081044 | 0.051225 |
| | | | NO: 280 | | | |

*Nucleotide sequences (nt) further comprise a stop codon (“taa”) at the end of each sequence, which is not shown.

### Example 2. Production and Characterization of Mutant CYP5491 Fusions

Signal peptide and point mutation strategies were combined to determine whether mutant CYP5491 fusions had increased C11 hydroxylase activity relative to wild-type CYP5491 fusions and mutant CYP5491 proteins alone. In the C11 hydroxylase screen described in Example 1, >80% of the hits were from active site single mutants and signal peptide optimization designs. The top two signal peptide fusions and the top three point mutations were combined to generate six new mutant fusions. Each mutant fusion was tested separately in the base host cell strain described in Example 1. Multiple copies of each mutant fusion were integrated into the genome of the host cell strain. The amino acid and nucleotide sequences of the mutant CYP5491 fusions are shown in Table 5 below. In Table 5, signal peptide “|P53903|PGA2|Processing of GAS1 and ALP protein 2|d23-129” denotes that the signal peptide of PGA2 (residues 1-22 of PGA2 (UniprotKB Accession No. P53903) was used in the fusion, while signal peptide “|Q07451|YET3|Endoplasmic reticulum transmembrane protein 3|d7-203” denotes that the signal peptide of YET3 (residues 1-6 of YET3 (UniprotKB Accession No. Q07451)) was used in the fusion. The amino acid substitution in the “Fusion” column of Table 5 denotes the amino acid substitution at a residue corresponding to the indicated amino acid in wild-type CYP5491 (SEQ ID NO: 208).

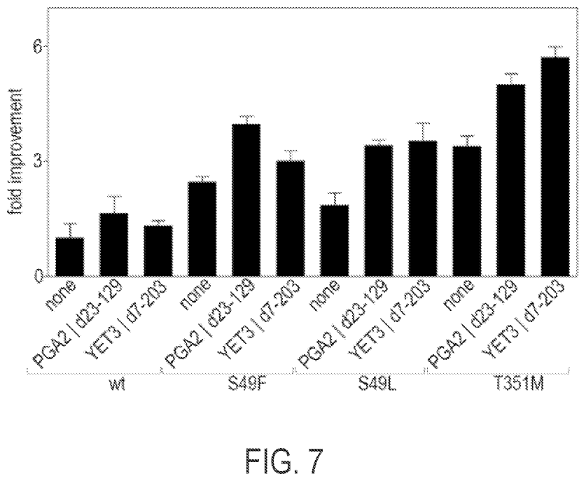

FIG. 7 shows that the signal peptide of PGA2 or YET3 further improved the activity of a CYP5491 protein that contained the point mutation T351M.

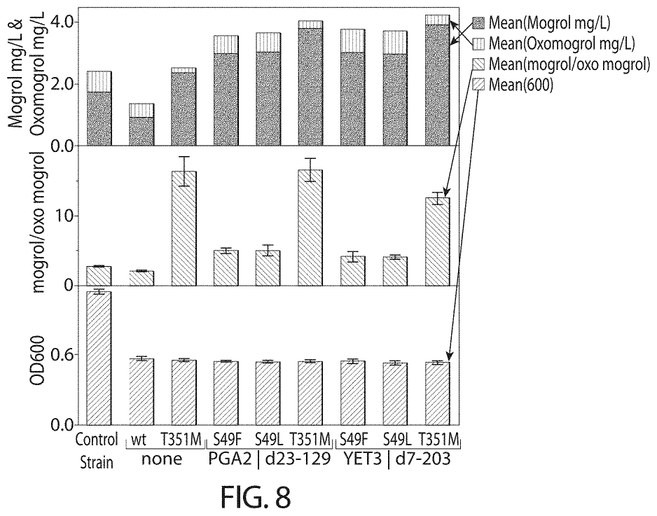

Since the C11 hydroxylase base strain-3 had a higher flux to mogrol than base strain-1, base strain-2, or a control strain, the C11 hydroxylase base strain-3 was used to determine if the point mutations or signal peptides changed the specificity of CYP5491. As shown in FIG. 8, the point mutation T351M increased the ratio of mogrol/oxo mogrol, indicating that the point mutation T351M results in improved activity and specificity to produce mogrol.

A mutant P450 fusion comprising PGA2 d 23-129/YET3 d 7-203 signal peptides with T351M in the background of base strain-3 produced mogrol titers that were about two times higher than a plasmid-bearing control strain in a 96-well plate assay. The control strain comprises a CDS, two EPHs, a C11-hydroxylase, two cytochrome P450 reductases, and an upregulated SQE.

### Example 3: Combining Recombinant Proteins to Produce a Mogrol Precursor, Mogrol, or a Mogroside

The recombinant proteins of the present disclosure are used in combination to produce a mogrol precursor, (e.g., 2-3-oxidosqualene, 2,3,22,23-dioxidosqualene, cucurbitadienol, 24, 25-expoxycucurbitadienol, 24,25-dihydroxycucurbitadienol), mogrol, or mogrosides (e.g., mogroside I-A1 (MIA1), mogroside I-E (MIE), mogroside II-A1 (MIIA1), mogroside III-A1 (MIIIA1), mogroside II-E (MIIE), mogroside III (MIII), siamenoside I, mogroside IV, mogroside III-E (MIIIE), mogroside V, and mogroside VI).

For example, to produce mogrol, genes encoding enzymes such as an SQE, a CDS, an EPH and a C11 hydroxylase are expressed in yeast cells. In some instances, a cytochrome P450 reductase is also expressed in the yeast cells. Non-limiting examples of suitable SQEs, EPHs, C11 hydroxylases and cytochrome P450 reductases are provided in Tables 4-7. Non-limiting examples of CDSs are provided in Table 8. Mogrol can be quantified using LC-MS. UGTs are further expressed in the yeast cells to produce mogrosides. Non-limiting examples of UGTs are provided in Table 9.

Alternatively, the recombinant proteins are purified from host cells and the mogrol is produced outside of the host cells. The recombinant proteins are added either sequentially or simultaneously to a reaction buffer comprising squalene.

### Example 4: Nucleic Acid and Protein Sequences Associated with the Disclosure

TABLE 5
Amino acid and nucleotide sequences
of mutant CYP5491 fusions

| | Amino Acid | Nucleotide |
|---|---|---|
| Fusion | Sequence | Sequence |
| signal peptide \| P53903 | SEQ ID | SEQ ID |
| \| PGA2 \| Processing of | NO: 305 | NO: 311 |
| GAS1 and ALP protein | | |
| 2 \| d23-129 \| T351M | | |
| signal peptide \| P53903 | SEQ ID | SEQ ID |
| \| PGA2 \| Processing of | NO: 306 | NO: 312 |
| GAS1 and ALP protein | | |
| 2 \| d23-129 \| S49L | | |
| signal peptide \| P53903 | SEQ ID | SEQ ID |
| \| PGA2 \| Processing of | NO: 307 | NO: 313 |
| GAS1 and ALP protein | | |
| 2 \| d23-129 \| S49F | | |
| signal peptide \| Q07451 | SEQ ID | SEQ ID |
| \| YET3 \| Endoplasmic | NO: 308 | NO: 314 |
| reticulum | | |
| transmembrane protein | | |
| 3 \| d7-203 \| T351M | | |
| signal peptide \| Q07451 | SEQ ID | SEQ ID |
| \| YET3 \| Endoplasmic | NO: 309 | NO: 315 |
| reticulum | | |
| transmembrane protein | | |
| 3 \| d7-203 \| S49L | | |
| signal peptide \| Q07451 | SEQ ID | SEQ ID |
| \| YET3 \| Endoplasmic | NO: 310 | NO: 316 |
| reticulum | | |
| transmembrane protein | | |
| 3 \| d7-203 \| S49F | | |

TABLE 6
Sequences of additional enzymes
associated with the disclosure

| | | Amino acid |
|---|---|---|
| Name | DNA sequences | sequences |
| CYP1798 | SEQ ID NO: 397 | SEQ ID NO: 406 |
| AtCPR1 | SEQ ID NO: 398 | SEQ ID NO: 407 |
| CPR4497 | SEQ ID NO: 399 | SEQ ID NO: 408 |
| sgCDS | SEQ ID NO: 400 | SEQ ID NO: 409 |
| EPH3 | SEQ ID NO: 401 | SEQ ID NO: 410 |
| atEPH2 | SEQ ID NO: 402 | SEQ ID NO: 411 |
| ERG9 | SEQ ID NO: 403 | SEQ ID NO: 412 |
| ERG1 | SEQ ID NO: 404 | SEQ ID NO: 413 |
| ERG7 | SEQ ID NO: 405 | SEQ ID NO: 414 |

TABLE 7
Additional C11 hydroxylase (P450), cytochrome
P450 reductase, EPH, and SQE sequences

| | Nucleotide | Amino Acid |
|---|---|---|
| Enzyme | Sequence | Sequence |
| C11 hydroxylase | SEQ ID NO: 113 | SEQ ID NO: 129 |
| C11 hydroxylase | SEQ ID NO: 114 | SEQ ID NO: 130 |
| (cucurbitadienol | | |
| oxidase) | | |
| Cytochrome P450 | SEQ ID NO: 115 | SEQ ID NO: 131 |
| reductase | | |
| Cytochrome P450 | SEQ ID NO: 116 | SEQ ID NO: 132 |
| reductase | | |
| Epoxide hydrolase | SEQ ID NO: 117 | SEQ ID NO: 133 |
| Epoxide hydrolase | SEQ ID NO: 118 | SEQ ID NO: 134 |
| Epoxide hydrolase | SEQ ID NO: 119 | SEQ ID NO: 135 |
| (epoxide hydratase) | | |
| Epoxide hydrolase | SEQ ID NO: 120 | SEQ ID NO: 136 |
| (epoxide hydratase) | | |
| Epoxide hydrolase | SEQ ID NO: 121 | SEQ ID NO: 137 |
| (epoxide hydratase) | | |
| Epoxide hydrolase | SEQ ID NO: 122 | SEQ ID NO: 138 |
| (epoxide hydratase) | | |
| Epoxide hydrolase | SEQ ID NO: 123 | SEQ ID NO: 139 |
| (epoxide hydratase) | | |
| Epoxide hydrolase | SEQ ID NO: 124 | SEQ ID NO: 140 |
| (epoxide hydratase) | | |
| Epoxide hydrolase | SEQ ID NO: 125 | SEQ ID NO: 141 |
| (epoxide hydratase) | | |
| Squalene epoxidase | SEQ ID NO: 126 | SEQ ID NO: 142 |
| Squalene epoxidase | SEQ ID NO: 127 | SEQ ID NO: 143 |
| Squalene epoxidase | SEQ ID NO: 128 | SEQ ID NO: 144 |

TABLE 8
Non-limiting examples of CDS sequences

| | Nucleotide | Amino Acid |
|---|---|---|
| Name | Sequence | Sequence |
| A0A0K9RW03_m | SEQ ID NO: 1 | SEQ ID NO: 41 |
| AquAgaCDS1_m | SEQ ID NO: 2 | SEQ ID NO: 42 |
| AquAgaCDS16 | SEQ ID NO: 3 | SEQ ID NO: 43 |
| AquAgaCDS6 | SEQ ID NO: 4 | SEQ ID NO: 44 |
| BenHisCDS2_m | SEQ ID NO: 5 | SEQ ID NO: 45 |
| A0A0D3QY32 | SEQ ID NO: 6 | SEQ ID NO: 46 |
| A0A0D3QXV2 | SEQ ID NO: 7 | SEQ ID NO: 47 |
| CmaCh17G013880.1 | SEQ ID NO: 8 | SEQ ID NO: 48 |
| A0A1S3CBF6 | SEQ ID NO: 9 | SEQ ID NO: 49 |
| CocGraCDS4 | SEQ ID NO: 10 | SEQ ID NO: 50 |
| CocGraCDS6_m | SEQ ID NO: 11 | SEQ ID NO: 51 |
| CSPI06G07180.1 | SEQ ID NO: 12 | SEQ ID NO: 52 |
| CucFoeCDS | SEQ ID NO: 13 | SEQ ID NO: 53 |
| CucMelMakCDS5 | SEQ ID NO: 14 | SEQ ID NO: 54 |
| CucMetCDS | SEQ ID NO: 15 | SEQ ID NO: 55 |
| CucPepOviCDS1_m | SEQ ID NO: 16 | SEQ ID NO: 56 |
| CucPepOviCDS2 | SEQ ID NO: 17 | SEQ ID NO: 57 |
| CucPepOviCDS3 | SEQ ID NO: 18 | SEQ ID NO: 58 |
| CucPepOviCDS3_m | SEQ ID NO: 19 | SEQ ID NO: 59 |
| Cucsa.349060.1 | SEQ ID NO: 20 | SEQ ID NO: 60 |
| F6GYI4 | SEQ ID NO: 21 | SEQ ID NO: 61 |
| GynCarCDS1 | SEQ ID NO: 22 | SEQ ID NO: 62 |
| GynCarCDS4 | SEQ ID NO: 23 | SEQ ID NO: 63 |
| K7NBZ9 | SEQ ID NO: 24 | SEQ ID NO: 64 |
| LagSicCDS2 | SEQ ID NO: 25 | SEQ ID NO: 65 |
| Lus10014538.g_m | SEQ ID NO: 26 | SEQ ID NO: 66 |
| Lus10032146.g_m | SEQ ID NO: 27 | SEQ ID NO: 67 |
| MomChaCDS2 | SEQ ID NO: 28 | SEQ ID NO: 68 |
| MomChaCDS4 | SEQ ID NO: 29 | SEQ ID NO: 69 |
| O23909_PEA_Y118L | SEQ ID NO: 30 | SEQ ID NO: 70 |
| Q6BE24 | SEQ ID NO: 31 | SEQ ID NO: 71 |
| SecEduCDS | SEQ ID NO: 32 | SEQ ID NO: 72 |
| SgCDS1 | SEQ ID NO: 33 | SEQ ID NO: 73 |
| SgCDS_Scer1 | SEQ ID NO: 34 | SEQ ID NO: 74 |
| TriKirCDS10 | SEQ ID NO: 35 | SEQ ID NO: 75 |
| TriKirCDS4 | SEQ ID NO: 36 | SEQ ID NO: 76 |
| XP_006340479.1 | SEQ ID NO: 37 | SEQ ID NO: 77 |
| XP_008655662.1 | SEQ ID NO: 38 | SEQ ID NO: 78 |
| XP_010541955.1_m | SEQ ID NO: 39 | SEQ ID NO: 79 |
| XP_016688836.1_m | SEQ ID NO: 40 | SEQ ID NO: 80 |

TABLE 9
Non-limiting examples of UGT sequences

| | Nucleotide | Amino acid |
|---|---|---|
| UGT name | sequence | sequence |
| | SEQ ID NO: 81 | SEQ ID NO: 97 |
| | SEQ ID NO: 82 | SEQ ID NO: 98 |
| UGT1697 | SEQ ID NO: 83 | SEQ ID NO: 99 |
| UGT720-269-4 | SEQ ID NO: 84 | SEQ ID NO: 100 |
| Q6VAA9_STERE | SEQ ID NO: 85 | SEQ ID NO: 101 |
| (Ntrunc(3) H49D L130W | | |
| P151K I159M V198L | | |
| A304T L421S A492T) | | |
| (D45H W126L K147P | | |
| M155I L194V T300A | | |
| S417L T488A) | | |
| U73C5_ARATH | SEQ ID NO: 86 | SEQ ID NO: 102 |
| U73C6_ARATH | SEQ ID NO: 87 | SEQ ID NO: 103 |
| UGT73-251.5 | SEQ ID NO: 88 | SEQ ID NO: 104 |
| UGT430 | SEQ ID NO: 89 | SEQ ID NO: 105 |
| Q6VAB4_STERE | SEQ ID NO: 90 | SEQ ID NO: 106 |
| | SEQ ID NO: 91 | SEQ ID NO: 107 |
| Q6VAA4_STERE | SEQ ID NO: 92 | SEQ ID NO: 108 |
| UGT94-289-1 | SEQ ID NO: 93 | SEQ ID NO: 109 |
| | SEQ ID NO: 94 | SEQ ID NO: 110 |
| | SEQ ID NO: 95 | SEQ ID NO: 111 |
| | SEQ ID NO: 96 | SEQ ID NO: 112 |

### EQUIVALENTS

Those skilled in the art will recognize, or be able to ascertain using no more than routine experimentation, many equivalents to the specific embodiments of the invention described in this application. Such equivalents are intended to be encompassed by the following claims.

All references, including patent documents, disclosed in this application are incorporated by reference in their entirety, particularly for the disclosure referenced in this application.

It should be appreciated that sequences disclosed in this application may or may not contain secretion signals. The sequences disclosed in this application encompass versions with or without secretion signals. It should also be understood that protein sequences disclosed in this application may be depicted with or without a start codon (M). The sequences disclosed in this application encompass versions with or without start codons. Accordingly, in some instances amino acid numbering may correspond to protein sequences containing a start codon, while in other instances, amino acid numbering may correspond to protein sequences that do not contain a start codon. It should also be understood that sequences disclosed in this application may be depicted with or without a stop codon. The sequences disclosed in this application encompass versions with or without stop codons. Aspects of the disclosure encompass host cells comprising any of the sequences described in this application and fragments thereof.

What is claimed is:

1. A host cell that expresses a C11 hydroxylase fusion protein, wherein the C11 hydroxylase fusion protein comprises:
a) a signal sequence that comprises
(i) a sequence selected from SEQ ID NO: 226, SEQ ID NO: 220 or SEQ ID NOs: 209-219, 221-225, or 227-232; or
(ii) a sequence that has no more than two amino acid substitutions, deletions, or insertions relative to a sequence selected from SEQ ID NO: 226, SEQ ID NO: 220, or SEQ ID NOs: 209-219, 221-225, or 227-232; and
b) a sequence comprising a transmembrane domain and a catalytic domain of a C11 hydroxylase enzyme.

2. The host cell of claim 1, wherein the signal sequence comprises a sequence selected from SEQ ID NO: 226, SEQ ID NO: 220, or SEQ ID NOs: 209-219, 221-225, or 227-232.

3. The host cell of claim 1 or 2, wherein the sequence in (b) comprises a sequence that is at least 90% identical to wild-type CYP5491 (SEQ ID NO: 208).

4. The host cell of any one of claims 1-3, wherein the transmembrane domain of the C11 hydroxylase enzyme comprises residues that correspond to residues 2-28 of wild-type CYP5491 (SEQ ID NO: 208).

5. The host cell of any one of claims 1-4, wherein the sequence comprising the catalytic domain of the C11 hydroxylase enzyme comprises an amino acid substitution, deletion, or insertion in the catalytic domain relative to the catalytic domain of wild-type CYP5491 (residues 29-473 of SEQ ID NO: 208).

6. The host cell of claim 5, wherein the amino acid substitution, deletion, or insertion is located in the substrate binding domain of the C11 hydroxylase enzyme.

7. The host cell of claim 6, wherein the amino acid substitution, deletion, or insertion is located in a loop that binds a heme group.

8. The host cell of any one of claims 1-7, wherein relative to the sequence of wild-type CYP5491 (SEQ ID NO: 208), the C11 hydroxylase enzyme comprises an amino acid substitution at a residue corresponding to residues: S49; V57; L76; A85; D107; L109; F112; T117; W119; L120; A140; F147; S155; H160; K185; L210; S211; L212; A282; D299; V350; T351; A353; L354; M376; I458; and/or T470 in CYP5491.

9. The host cell of claim 8, wherein the C11 hydroxylase enzyme comprises:
a) A, F, H, I, M, or L at the residue corresponding to S49 of wild-type CYP5491 (SEQ ID NO: 208);
b) A at the residue corresponding to V57 of wild-type CYP5491 (SEQ ID NO: 208);
c) I or V at the residue corresponding to L76 of wild-type CYP5491 (SEQ ID NO: 208);
d) S at the residue corresponding to A85 of wild-type CYP5491 (SEQ ID NO: 208);
e) P or R at the residue corresponding to D107 of wild-type CYP5491 (SEQ ID NO: 208);
f) A, C, F, W, or Y at the residue corresponding to L109 of wild-type CYP5491 (SEQ ID NO: 208);
g) T or W at the residue corresponding to F112 of wild-type CYP5491 (SEQ ID NO: 208);
h) G at the residue corresponding to T117 of wild-type CYP5491 (SEQ ID NO: 208);
i) R at the residue corresponding to W119 of wild-type CYP5491 (SEQ ID NO: 208);
j) H or N at the residue corresponding to L120 of wild-type CYP5491 (SEQ ID NO: 208);
k) P at the residue corresponding to A140 of wild-type CYP5491 (SEQ ID NO: 208);
l) L at the residue corresponding to F147 of wild-type CYP5491 (SEQ ID NO: 208);
m) A at the residue corresponding to S155 of wild-type CYP5491 (SEQ ID NO: 208);
n) E at the residue corresponding to H160 of wild-type CYP5491 (SEQ ID NO: 208);
o) H at the residue corresponding to K185 of wild-type CYP5491 (SEQ ID NO: 208);
p) S at the residue corresponding to L210 of wild-type CYP5491 (SEQ ID NO: 208);
q) N at the residue corresponding to S211 of wild-type CYP5491 (SEQ ID NO: 208);
r) F at the residue corresponding to L212 of wild-type CYP5491 (SEQ ID NO: 208);
s) V at the residue corresponding to A282 of wild-type CYP5491 (SEQ ID NO: 208);
t) A at the residue corresponding to D299 of wild-type CYP5491 (SEQ ID NO: 208);
u) F, I, L, or M at the residue corresponding to V350 of wild-type CYP5491 (SEQ ID NO: 208);
v) L or M at the residue corresponding to T351 of wild-type CYP5491 (SEQ ID NO: 208);
w) G at the residue corresponding to A353 of wild-type CYP5491 (SEQ ID NO: 208);
x) V or I at the residue corresponding to L354 of wild-type CYP5491 (SEQ ID NO: 208);
y) A or C at the residue corresponding to M376 of wild-type CYP5491 (SEQ ID NO: 208);
z) P at the residue corresponding to I458 of wild-type CYP5491 (SEQ ID NO: 208); and/or
aa) E at the residue corresponding to T470 of wild-type CYP5491 (SEQ ID NO: 208).

10. The host cell of any one of claims 1-9, wherein the host cell further comprises an upregulated squalene epoxidase, at least one cytochrome P450 reductase, at least one cucurbitadienol synthase (CDS), and/or at least one epoxide hydrolase (EPH).

11. The host cell of claim 10, wherein the host cell comprises an upregulated squalene synthase, a downregulated lanosterol synthase, at least one other C11 hydroxylase, and/or at least two cytochrome P450 reductases.

12. The host cell of any one of claims 1-11, wherein a nucleotide sequence encoding the C11 hydroxylase fusion protein is integrated into the genome of the host cell.

13. The host cell of any one of claims 1-12, wherein a nucleotide sequence encoding the C11 hydroxylase fusion protein is expressed on a plasmid.

14. The host cell of claim 12, wherein multiple copies of a nucleotide sequence encoding the C11 hydroxylase fusion protein are integrated into the genome of the host cell.

15. The host cell of any one of claims 10-14, wherein at least one nucleotide sequence encoding the squalene synthase, the squalene epoxidase, the at least one other C11 hydroxylase, the at least one cytochrome P450 reductase, the at least one CDS, and/or the at least one EPH is integrated into the genome of the host cell.

16. The host cell of any one of claims 10-15, wherein the host cell produces mogrol.

17. The host cell of any one of claims 10-16, wherein the host cell produces at least 1.1 fold more mogrol compared to a control host cell, wherein the control host cell comprises wild-type CYP5491.

18. The host cell of claim 16 or 17, wherein the host cell is capable of being cultured in cell culture media that is substantially free of one or more mogrol precursors that are not produced by the host cell.

19. The host cell of any one of claims 16-18, wherein the host cell is capable of producing a ratio of mogrol/11-oxomogrol that is greater than 2.

20. The host cell of any one of claims 17-19, wherein the C11 hydroxylase fusion protein comprises a sequence that is at least 90% identical to a sequence selected from SEQ ID NO: 305 or 308, SEQ ID NOs: 257-280, or SEQ ID NOs: 306-307, or SEQ ID NOs: 309-310.

21. A host cell that comprises a C11 hydroxylase enzyme, wherein the C11 hydroxylase enzyme is at least 90% identical to wild-type CYP5491 (SEQ ID NO: 208) and comprises an amino acid substitution at one or more residues corresponding to residues: S49; V57; L76; A85; D107; L109; F112; T117; W119; L120; A140; F147; S155; H160; K185; L210; S211; L212; A282; D299; V350; T351; A353; L354; M376; I458; and/or T470 in CYP5491.

22. The host cell of claim 21, wherein the C11 hydroxylase enzyme comprises:
a) A, F, H, I, M, or L at the residue corresponding to S49 of wild-type CYP5491 (SEQ ID NO: 208);
b) A at the residue corresponding to V57 of wild-type CYP5491 (SEQ ID NO: 208);
c) I or V at the residue corresponding to L76 of wild-type CYP5491 (SEQ ID NO: 208);
d) S at the residue corresponding to A85 of wild-type CYP5491 (SEQ ID NO: 208);
e) P or R at the residue corresponding to D107 of wild-type CYP5491 (SEQ ID NO: 208);
f) A, C, F, W, or Y at the residue corresponding to L109 of wild-type CYP5491 (SEQ ID NO: 208);
g) T or W at the residue corresponding to F112 of wild-type CYP5491 (SEQ ID NO: 208);
h) G at the residue corresponding to T117 of wild-type CYP5491 (SEQ ID NO: 208);
i) R at the residue corresponding to W119 of wild-type CYP5491 (SEQ ID NO: 208);
j) H or N at the residue corresponding to L120 of wild-type CYP5491 (SEQ ID NO: 208);
k) P at the residue corresponding to A140 of wild-type CYP5491 (SEQ ID NO: 208);
l) L at the residue corresponding to F147 of wild-type CYP5491 (SEQ ID NO: 208);
m) A at the residue corresponding to S155 of wild-type CYP5491 (SEQ ID NO: 208);
n) E at the residue corresponding to H160 of wild-type CYP5491 (SEQ ID NO: 208);
o) H at the residue corresponding to K185 of wild-type CYP5491 (SEQ ID NO: 208);
p) S at the residue corresponding to L210 of wild-type CYP5491 (SEQ ID NO: 208);
q) N at the residue corresponding to S211 of wild-type CYP5491 (SEQ ID NO: 208);
r) F at the residue corresponding to L212 of wild-type CYP5491 (SEQ ID NO: 208);
s) V at the residue corresponding to A282 of wild-type CYP5491 (SEQ ID NO: 208);
t) A at the residue corresponding to D299 of wild-type CYP5491 (SEQ ID NO: 208);
u) F, I, L, or M at the residue corresponding to V350 of wild-type CYP5491 (SEQ ID NO: 208);
v) L or M at the residue corresponding to T351 of wild-type CYP5491 (SEQ ID NO: 208);
w) G at the residue corresponding to A353 of wild-type CYP5491 (SEQ ID NO: 208);
x) V or I at the residue corresponding to L354 of wild-type CYP5491 (SEQ ID NO: 208);
y) A or C at the residue corresponding to M376 of wild-type CYP5491 (SEQ ID NO: 208);
z) P at the residue corresponding to I458 of wild-type CYP5491 (SEQ ID NO: 208); and/or
aa) E at the residue corresponding to T470 of wild-type CYP5491 (SEQ ID NO: 208).

23. The host cell of claim 22, wherein the C11 hydroxylase enzyme comprises:
a) a phenylalanine (F) or leucine (L) at a residue corresponding to S49 in wild-type CYP5491 (SEQ ID NO: 208); and/or
b) a methionine (M) at a residue corresponding to T351 in wild-type CYP5491 (SEQ ID NO: 208).

24. The host cell of any one of claims 21-23, wherein the C11 hydroxylase enzyme is expressed as a C11 hydroxylase fusion protein that comprises a signal sequence that is at least 90% identical to a sequence selected from SEQ ID NO: 226, SEQ ID NO: 220 or SEQ ID NOs: 209-219, 221-225, or 227-232.

25. The host cell of claim 24, wherein the signal sequence comprises a sequence selected from SEQ ID NO: 226, SEQ ID NO: 220 or SEQ ID NOs: 209-219, 221-225, or 227-232.

26. The host cell of any one of claims 21-25, wherein the host cell further comprises an upregulated squalene epoxidase, at least one cytochrome P450 reductase, at least one cucurbitadienol synthase (CDS), and/or at least one epoxide hydrolase (EPH).

27. The host cell of claim 26, wherein the host cell comprises an upregulated squalene synthase, a downregulated lanosterol synthase, at least one other C11 hydroxylase, and/or at least two cytochrome P450 reductases.

28. The host cell of any one of claims 21-27, wherein a nucleotide sequence encoding the C11 hydroxylase enzyme is integrated into the genome of the host cell or a nucleotide sequence encoding the C11 hydroxylase enzyme is expressed on a plasmid.

29. The host cell of claim 28, wherein multiple copies of a nucleotide sequence encoding the C11 hydroxylase enzyme are integrated into the genome of the host cell.

30. The host cell of any one of claims 27-29, wherein at least one nucleotide sequence encoding the squalene synthase, the squalene epoxidase, the at least one other C11 hydroxylase, the at least one cytochrome P450 reductase, the at least one CDS, and/or the at least one EPH is integrated into the genome of the host cell.

31. The host cell of any one of claims 26-30, wherein the host cell produces mogrol.

32. The host cell of any one of claims 26-31, wherein the host cell produces 1.1 fold more mogrol compared to a control host cell, wherein the control host cell comprises wild-type CYP5491.

33. The host cell of any one of claims 26-32, wherein the host cell is capable of being cultured in cell culture media that is substantially free of one or more mogrol precursors that are not produced by the host cell.

34. The host cell of any one of claims 31-33, wherein the host cell is capable of producing a ratio of mogrol/11-oxomogrol that is greater than 2.

35. A method of producing mogrol comprising culturing the host cell of any one of claims 1-34.

36. The method of claim 35, wherein the host cell is cultured in the presence of a mogrol precursor selected from squalene, 2-3-oxidosqualene, cucurbitadienol, 2-3, 22,23-diepoxysqualene, 24, 25 epoxy-cucurbitadienol, and 24, 25 dihydroxy cucurbitadienol.

37. The method of claim 35, wherein the host cell is cultured in media that is substantially free of one or more mogrol precursors that are not produced by the host cell.

38. A host cell that comprises a C11 hydroxylase fusion protein, wherein the C11 hydroxylase fusion protein comprises the signal sequence of ERG11 and a sequence encoding a transmembrane domain and a catalytic domain of a C11 hydroxylase enzyme.

39. The host cell of claim 38, wherein the C11 hydroxylase fusion protein comprises residues 3-504 of wild-type CYP5491 (SEQ ID NO: 208).

40. The host cell of claim 38 or 39, wherein the C11 hydroxylase fusion protein comprises a sequence that is at least 90% identical to SEQ ID NO: 280.

41. A C11 hydroxylase fusion protein, wherein the fusion protein comprises:
a) a signal sequence that is at least 90% identical to a sequence selected from SEQ ID NO: 226, SEQ ID NO: 220 or SEQ ID NOs: 209-219, 221-225, or 227-232, and a sequence encoding a transmembrane domain and a catalytic domain of a C11 hydroxylase enzyme; or
b) the first 25 amino acids of ERG11, and a sequence encoding a transmembrane domain and a catalytic domain of a C11 hydroxylase enzyme.

42. A method of producing mogrol comprising contacting a C11 hydroxylase enzyme with:
(a) 24, 25 dihydroxy cucurbitadienol, thereby producing mogrol;
(b) cucurbitadienol, thereby producing 11-hydroxycucurbitadienol; and/or
(c) 24,25-epoxy cucurbitadienol, thereby producing, 11-hydroxy-24,25-epoxycucurbitadienol,
wherein the C11 hydroxylase enzyme is at least 90% identical to SEQ ID NO: 208 and comprises at least one amino acid substitution relative to SEQ ID NO: 208.

43. The method of claim 42, wherein relative to the sequence of wild-type CYP5491 (SEQ ID NO: 208), the C11 hydroxylase enzyme comprises an amino acid substitution at a residue corresponding to residues: S49; V57; L76; A85; D107; L109; F112; T117; W119; L120; A140; F147; S155; H160; K185; L210; S211; L212; A282; D299; V350; T351; A353; L354; M376; I458; and/or T470 in wild-type CYP5491 (SEQ ID NO: 208).

44. The host cell of claim 42 or 43, wherein the C11 hydroxylase enzyme comprises:
a) A, F, H, I, M, or L at the residue corresponding to S49 in wild-type CYP5491 (SEQ ID NO: 208);
b) A at the residue corresponding to V57 in wild-type CYP5491 (SEQ ID NO: 208);
c) I or V at the residue corresponding to L76 in wild-type CYP5491 (SEQ ID NO: 208);
d) S at the residue corresponding to A85 in wild-type CYP5491 (SEQ ID NO: 208);
e) P or R at the residue corresponding to D107 in wild-type CYP5491 (SEQ ID NO: 208);
f) A, C, F, W, or Y at the residue corresponding to L109 in wild-type CYP5491 (SEQ ID NO: 208);
g) T or W at the residue corresponding to F112 in wild-type CYP5491 (SEQ ID NO: 208);
h) G at the residue corresponding to T117 in wild-type CYP5491 (SEQ ID NO: 208);
i) R at the residue corresponding to W119 in wild-type CYP5491 (SEQ ID NO: 208);
j) H or N at the residue corresponding to L120 in wild-type CYP5491 (SEQ ID NO: 208);
k) P at the residue corresponding to A140 in wild-type CYP5491 (SEQ ID NO: 208);
l) L at the residue corresponding to F147 in wild-type CYP5491 (SEQ ID NO: 208);
m) A at the residue corresponding to S155 in wild-type CYP5491 (SEQ ID NO: 208);
n) E at the residue corresponding to H160 in wild-type CYP5491 (SEQ ID NO: 208);
o) H at the residue corresponding to K185 in wild-type CYP5491 (SEQ ID NO: 208);
p) S at the residue corresponding to L210 in wild-type CYP5491 (SEQ ID NO: 208);
q) N at the residue corresponding to S211 in wild-type CYP5491 (SEQ ID NO: 208);
r) F at the residue corresponding to L212 in wild-type CYP5491 (SEQ ID NO: 208);
s) V at the residue corresponding to A282 in wild-type CYP5491 (SEQ ID NO: 208);
t) A at the residue corresponding to D299 in wild-type CYP5491 (SEQ ID NO: 208);
u) F, I, L, or M at the residue corresponding to V350 in wild-type CYP5491 (SEQ ID NO: 208);
v) L or M at the residue corresponding to T351 in wild-type CYP5491 (SEQ ID NO: 208);
w) G at the residue corresponding to A353 in wild-type CYP5491 (SEQ ID NO: 208);
x) V or I at the residue corresponding to L354 in wild-type CYP5491 (SEQ ID NO: 208);
y) A or C at the residue corresponding to M376 in wild-type CYP5491 (SEQ ID NO: 208);
z) P at the residue corresponding to I458 in wild-type CYP5491 (SEQ ID NO: 208); and/or
aa) E at the residue corresponding to T470 in wild-type CYP5491 (SEQ ID NO: 208).

45. The method of any one of claims 42-44, wherein the C11 hydroxylase enzyme is a purified protein.

46. A host cell that comprises a C11 hydroxylase fusion protein, wherein the C11 hydroxylase fusion protein comprises:
a) the signal sequence of KAR2, NCP1, ERP2, RBD2, SNA3, SPC2, NHX1, PGA2, GRX6, YLR413W, YJL062W, MSC2, EMCS, CHO2, IFA38, SUR2, IPT1, YET3, YPL162C, ERG11, SRP102, GUP1, CBR1, or YHR138C; and
b) a sequence encoding a transmembrane domain and a catalytic domain of a C11 hydroxylase enzyme.

47. A host cell that comprises a C11 hydroxylase fusion protein, wherein the C11 hydroxylase fusion protein is at least 90% identical to any one of SEQ ID NO: 305 or 308, SEQ ID NOs: 257-280, or SEQ ID NOs: 306-307, or SEQ ID NOs: 309-310.

48. The host cell of claim 47, wherein the C11 hydroxylase fusion protein is at least 98% identical to any one of SEQ ID NO: 305 or 308, SEQ ID NOs: 257-280, or SEQ ID NOs: 306-307, or SEQ ID NOs: 309-310.

49. The host cell of claim 47 or 48, wherein the C11 hydroxylase fusion protein is at least 99% identical to any one of SEQ ID NO: 305 or 308, SEQ ID NOs: 257-280, or SEQ ID NOs: 306-307, or SEQ ID NOs: 309-310.

50. The host cell of any one of claims 47-49, wherein the C11 hydroxylase fusion protein comprises any one of SEQ ID NO: 305 or 308, SEQ ID NOs: 257-280, or SEQ ID NOs: 306-307, or SEQ ID NOs: 309-310.

51. The host cell of claim 50, wherein the sequence is PGA2Id23-129_CYP5491-T351M (SEQ ID NO: 305).

52. A method comprising culturing the host cell of any one of claims 46-51.

53. The host cell of any one of claims 1-20, wherein the catalytic domain of the C11 hydroxylase enzyme comprises residues that correspond to residues 29-473 of wild-type CYP5491 (SEQ ID NO: 208).
